(19) United States
(12) Patent Application Publication
(10) Pub. No.: US 2023/0039437 A1
(43) Pub. Date: Feb. 9, 2023

(54) Soft Tissue Graft Preparations and Methods of Use

(71) Applicant: Stryker Corporation, Kalamazoo, MI (US)

(72) Inventors: James Genuario, Lone Tree, CO (US); John Schelter, Centennial, CO (US); Kelley E. Mccarroll, San Jose, CA (US); Kyle Craig Pilgeram, San Jose, CA (US); David Marucheau, Denver, CO (US); Harold D. Sampson, JR., Lone Tree, CO (US)

(73) Assignee: Stryker Corporation, Kalamazoo, MI (US)

(21) Appl. No.: 17/816,528

(22) Filed: Aug. 1, 2022

Related U.S. Application Data
(60) Provisional application No. 63/314,692, filed on Feb. 28, 2022.
(60) Provisional application No. 63/227,730, filed on Jul. 30, 2021.

(51) Int. Cl. A61F 2/08; A61B 17/04

(52) U.S. Cl. CPC A61F 2/0811; A61B 17/0401; A61F 2002/0882; A61B 2017/0464; A61F 2002/0858; A61F 2002/0852; A61F 2240/001; A61B 2017/00526

(57) ABSTRACT

In one embodiment, the present disclosure may be a method for preparing a soft tissue graft, comprising: passing a suture material through the soft tissue graft adjacent to a graft support filament at a first longitudinal location along the filament and graft, such that the suture material passes through the graft on a first side of the filament; passing the suture material over the filament and back through the graft adjacent to the filament at a second longitudinal location spaced longitudinally along the filament and graft from the first longitudinal location, such that the suture material passes through the graft on the first side of the filament; and tensioning the suture material to secure the filament to the graft along a length of the suture material between the first longitudinal location and the second longitudinal location.

### CROSS-REFERENCE PARAGRAPH

This application claims the benefit of the filing date of United States Provisional Application Nos. 63/314,692 filed on Feb. 28, 2022, and 63/227,730 filed on Jul. 30, 2021, the disclosures of which are hereby incorporated herein by reference.

### BACKGROUND OF THE INVENTION

The repair of soft tissue injuries comes in many forms, and can include variations from the repair of existing soft tissue to the replacement/reconstruction of soft tissue with a graft. The latter is typically required, for example, in the repair of tears in the ligaments of the knee, such as the anterior cruciate ligament (ACL) or the posterior cruciate ligament (PCL), where the complete replacement of soft tissue with a graft is often required for a successful repair. These soft tissue grafts can also vary, and can include allografts, autografts, xenografts, or artificial grafts.

Even within these various categories of grafts, there are still further decisions to be made as different graft materials and sources have different material properties, biomechanical capabilities, etc. One graft material recently gaining interest for use in ACL replacement is an allograft or autograft from a quadriceps tendon. This material is bulky, strong, relatively stiff, and has the desired biomechanical properties similar to that of a native ACL, particularly when compared to a patellar tendon graft. The quadriceps tendon is also relatively thick, which allows for it to be shaped to fill a greater volume of a bone tunnel without the need to double-over the graft material.

Conversely, due to the natural volume of the quadriceps tendon graft, surgical operators must consider options in preparing the graft for ACL replacement, particularly in deciding how to suspend the graft within the bone tunnels and across the joint space. For instance, when using a commonly used suspension device such as adjustable suture loops and suture button anchors, operators may have difficulty in coupling the suture loops to the graft material, commonly requiring additional sutures to be positioned between the graft and the loops. These additional loops, however, necessarily take up a portion of the length within the bone tunnels—even though they can be adjusted to become smaller loops, such existing suture constructs nevertheless occupy a length within the bone tunnel, which typically results in decreased surface area contact between the graft and the bone surrounding the tunnels. Thus, there exists a need to improve the preparation of such grafts to ensure maximum chances of a successful outcome. Further, the fixation of the suture loop to the graft is held only by a synthetic suture construct (e.g., the additional suture loops). Thus the possibility for creep between the suture construct and the graft exists, which could reduce the tension within the repair and thus a decreased success of a successful and long-term outcome. There exists a need to reduce creep and ensure maximum chances of a successful repair and outcome.

### BRIEF SUMMARY OF THE INVENTION

Generally, the present disclosure includes various methods of preparing and using soft tissue grafts, as well as various structures prepared by using such methods of preparation and use.

In one aspect, a method for preparing a soft tissue graft, comprising the steps of: passing a suture material through the soft tissue graft adjacent to a graft support filament at a first longitudinal location along the graft support filament and the soft tissue graft, such that the suture material passes through the soft tissue graft on a first side of the graft support filament; passing the suture material over the graft support filament and back through the soft tissue graft adjacent to the graft support filament at a second longitudinal location spaced longitudinally along the graft support filament and the soft tissue graft from the first longitudinal location, such that the suture material passes through the soft tissue graft on the first side of the graft support filament; and tensioning the suture material to secure the graft support filament to the soft tissue graft at the first longitudinal location and at the second longitudinal location.

Further, the suture material may include a needle, wherein during the passing steps the needle pierces the soft tissue graft to pass the suture material through the soft tissue graft. Moreover, the passing steps and tensioning step may be repeated as desired at one more additional longitudinal locations along the graft support filament and the soft tissue graft. Still further, the graft support filament may include a length of suture including at least one adjustable filamentary loop formed therein, wherein the first and second longitudinal locations are along a length of the at least one adjustable filamentary loop. Also, prior to the first passing step, the suture material may be aligned and secured to the graft support filament such that the suture material and the graft support filament are manipulated as a single device. Further, after the tensioning step, the graft support filament may be slidable relative to the soft tissue graft and the suture material.

In another aspect, a method of implanting a soft tissue graft construct in a bone tunnel formed in a bone, comprising the steps of: positioning a soft tissue graft construct within the bone tunnel, the soft tissue graft construct including a soft tissue graft and a graft support filament slidably secured to the soft tissue graft by a suture material, the suture material passing through the soft tissue graft at least twice and looping around the graft support filament at least once, and a fixation member connected to the graft support filament; positioning the fixation member relative to the bone to secure the graft support filament relative to the bone; and tensioning the graft support filament to position the soft tissue graft within the bone tunnel, wherein upon tensioning, at least a portion of the graft support filament slides relative to the soft tissue graft and the suture material.

Further, the graft support filament may be positioned alongside the soft tissue graft and includes at least one adjustable filamentary loop, and the suture material may be positioned to alternate between passing through the soft tissue graft and around the graft support filament in a spiral pattern along at least part of the length of the soft tissue graft and the graft support filament, such that during the tensioning step, the at least one adjustable filamentary loop of the graft support filament may slide alongside the soft tissue graft and through the spiral pattern of the suture material.

In yet another aspect, a soft tissue graft suspension system, comprising: a soft tissue graft; a graft support filament including at least one adjustable filamentary loop; and a suture material, wherein the at least part of at least one adjustable filamentary loop of the graft support filament is positioned alongside the soft tissue graft, and the suture material is positioned to alternate between passing through the soft tissue graft and around the at least one adjustable filamentary loop in a spiral pattern along at least part of the length of the soft tissue graft and the at least one adjustable filamentary loop, wherein the at least one adjustable filamentary loop is adapted to be slidable alongside the soft tissue graft and through the spiral pattern of the suture material.

Further, the at least one adjustable filamentary loop may be a loop formed by a splice in the body of the graft support filament and the at least one adjustable filamentary loop is associated with a fixation device, wherein the suture material may engage the graft support filament in a luggage-tag configuration at a location on the loop distant from the fixation device such that a first end and a second end of the suture material each extends from the luggage-tag configuration. Moreover, each of the first end and the second end of the suture material may include a first needle and a second needle, respectively, each needle adapted for piercing the soft tissue graft.

Still further, each of the first and second ends of the suture material may form a respective spiral pattern along at least part of the length of the soft tissue graft and the at least one filamentary loop by the first end having a spiral pattern along the at least one filamentary loop in a first direction along the at least one filamentary loop from the luggage-tag configuration towards the fixation device, and the second end having a spiral pattern along the at least one filamentary loop in a second direction along the at least one filamentary loop from the luggage-tag configuration towards the fixation device. The first direction may, for example, be a counterclockwise direction along the at least one filamentary loop from the luggage-tag configuration towards the fixation device, and the second direction may, for example, be a clockwise direction along the at least one filamentary loop from the luggage-tag configuration towards the fixation device.

In still a further aspect, a method for preparing a soft tissue graft, comprising the steps of: engaging a length of suture material to an at least one filamentary loop of a graft support filament, the at least one filamentary loop extending formed by a splice in a body of the graft support filament and the at least one adjustable filamentary loop is associated with a fixation device, wherein the suture material engages the graft support filament at a location on the loop distant from the fixation device such that a first end and a second end of the suture material extend from the at least one filamentary loop; passing both the first end and the second end of the suture material through the soft tissue graft at a first location away from both a first end and a second end of the soft tissue graft; tensioning both the first end and the second end of the suture material to position the at least one filamentary loop adjacent to the soft tissue graft; passing the first end of the suture material, sequentially, through the soft tissue graft and around the at least one filamentary loop in a first direction along the at least one filamentary loop from the first location towards the fixation device and towards the first end of the soft tissue graft; and passing the second end of the suture material, sequentially, through the soft tissue graft and around the at least one filamentary loop in a second direction along the at least one filamentary loop from the first location towards the fixation device and towards the first end of the soft tissue graft; wherein the at least one filamentary loop is slidable relative to the suture material and the soft tissue graft. The first direction may, for example, be a counterclockwise direction along the at least one filamentary loop from the first location towards the fixation device and towards the first end of the soft tissue graft, and the second direction may be, for example, a clockwise direction along the at least one filamentary loop from the first location towards the fixation device and towards the first end of the soft tissue graft.

Moreover, the suture material may engage the graft support filament at a location on the loop distant from the fixation device in a luggage-tag configuration such that a first end and a second end of the suture material extend from the at least one filamentary loop.

The method may further include: passing the first end of the suture material, sequentially, through the soft tissue graft and around the at least one filamentary loop in one of the first or second directions along the at least one filamentary loop from the fixation device towards the first location and away from the first end of the soft tissue graft; passing the second end of the suture material, sequentially, through the soft tissue graft and around the at least one filamentary loop in the other of the first or second directions along the at least one filamentary loop from the fixation device towards the first location and away from the first end of the soft tissue graft; and after the passing steps, securing the first and second ends of the suture material at a location adjacent to the first location. The first direction may be, for example, a counterclockwise direction along the at least one filamentary loop from the splice towards the first location and away from the first end of the soft tissue graft, and the second direction may be, for example, a clockwise direction along the at least one filamentary loop from the splice towards the first location and away from the first end of the soft tissue graft.

In yet another aspect, a method for preparing a soft tissue graft, including passing a first suture material through the soft tissue graft adjacent to a first graft support filament at a first longitudinal location along the first graft support filament and the soft tissue graft, such that the second suture material passes through the soft tissue graft on a first side of the first graft support filament; passing the first suture material over the first graft support filament and back through the soft tissue graft adjacent to the first graft support filament at a second longitudinal location spaced longitudinally along the first graft support filament and the soft tissue graft from the first longitudinal location, such that the first suture material passes through the soft tissue graft on the first side of the first graft support filament; and tensioning the first suture material to secure the first graft support filament to the soft tissue graft at the first longitudinal location and at the second longitudinal location.

The method may further include: passing a second suture material through the soft tissue graft adjacent to a second graft support filament at a third longitudinal location spaced longitudinally along the second graft support filament and the soft tissue graft from the first and second longitudinal locations, such that the second suture material passes through the soft tissue graft on a first side of the second graft support filament; passing the second suture material over the second graft support filament and back through the soft tissue graft adjacent to the second graft support filament at a fourth longitudinal location spaced longitudinally along the second graft support filament and the soft tissue graft from the first, second and third longitudinal locations, such that the second suture material passes through the soft tissue graft on the first side of the second graft support filament; and tensioning the second suture material to secure the second graft support filament to the soft tissue graft at the third longitudinal location and at the fourth longitudinal location.

In another aspect, a soft tissue graft construct, including a soft tissue graft having a longitudinal length, a first end and a second end; a first graft support filament; a second graft support filament; and at least a first length of suture material passing through the soft tissue graft in at least a first location and passing around but not through at least one of the first graft support filament and the second graft support filament.

The construct may further include a second length of suture material passing through the soft tissue graft in at least a second location different from the first location and passing around but not through at least one of the first graft support filament and the second graft support filament. Further, the first length of suture material may pass around the first graft support filament and not the second graft support filament, and the second length of suture material may pass around the second graft support filament and not the first graft support filament. Still further, the first length of suture material may be connected to the first graft support filament and the second length of suture material may be connected to the second graft support filament, or alternatively, the second length of suture material may be connected to the first graft support filament and the first length of suture material may be connected to the second graft support filament.

Still further, the first graft support filament may extend along the longitudinal length of the soft tissue graft towards and beyond the first end and the second graft support filament may extend along the longitudinal length of the soft tissue graft towards and beyond the second end. Moreover, each of the first and second graft support filament may include at least one filamentary loop, wherein the at least one filamentary loop of the first graft support filament is interconnected with the at least one filamentary loop of the second graft support filament.

In yet a further aspect, a method of preparing a soft tissue graft, including positioning a first graft support filament and a second graft support relative to the soft tissue graft, the soft tissue graft having a longitudinal length, a first end and a second end such that the first graft support filament extends along the longitudinal length from a location on the soft tissue graft, towards and beyond the first end of the soft tissue graft, and the second graft support filament extends along the longitudinal length from a location on the soft tissue graft, towards and beyond the second end of the soft tissue graft; passing a first suture material through the soft tissue graft in at least a first location and passing around but not through at least one of the first graft support filament and the second graft support filament; and passing a second suture material through the soft tissue graft in at least a second location different from the first location and passing around but not through at least one of the first graft support filament and the second graft support filament.

As to any of the examples herein, the soft tissue graft may be a portion of a quadriceps tendon.

### BRIEF DESCRIPTION OF THE DRAWINGS

FIGS. 1A-6C illustrate various elements of one embodiment for the preparation of a soft tissue graft.

FIGS. 7A-12E illustrate various elements of another embodiment for the preparation of a soft tissue graft.

FIGS. 13A-13G illustrate various further elements and examples of various preparations of a soft tissue graft.

FIGS. 14 and 14A illustrate various examples of other methods of preparing a soft tissue graft and positioning a graft support filament relative to a soft tissue graft.

FIGS. 15-19C illustrate various further elements and examples of various preparations of a soft tissue graft.

### DETAILED DESCRIPTION

As used herein unless stated otherwise, the term “proximal” means closer to the operator, and the term “distal” means further from the operator. As used herein, the terms “about,” “generally,” and “substantially” are intended to mean that slight deviations from absolute are included within the scope of the term so modified.

Many of the embodiments and examples discussed herein refer specifically to a quadriceps tendon graft (quad tendon graft), but any soft tissue graft made of any material or from any source may be used in place of the quad tendon graft. The quad tendon graft discussed in particular in the embodiments and examples herein may be autologous tissue (autograft) taken from the patient's quad tendon. Alternatively, the quad tendon graft may be from a donor (allograft), or may be from a donor of another species (xenograft). Alternative anatomical donor sites may include the patellar tendon, anterior tibialis or achilles, for example, or can be from another anatomical location. Of course, as known in the art, artificial materials may also be used to form a suitable soft tissue graft. Further, as illustrated herein, the graft material is entirely soft tissue, and thus the preparation methods disclosed herein can be applied to any portion of such a graft. However, another form of quadriceps tendon graft (and indeed, other types of grafts) may include a bone block or bone plug. With such grafts, the methods disclosed herein may be applied to the soft tissue portions of such grafts.

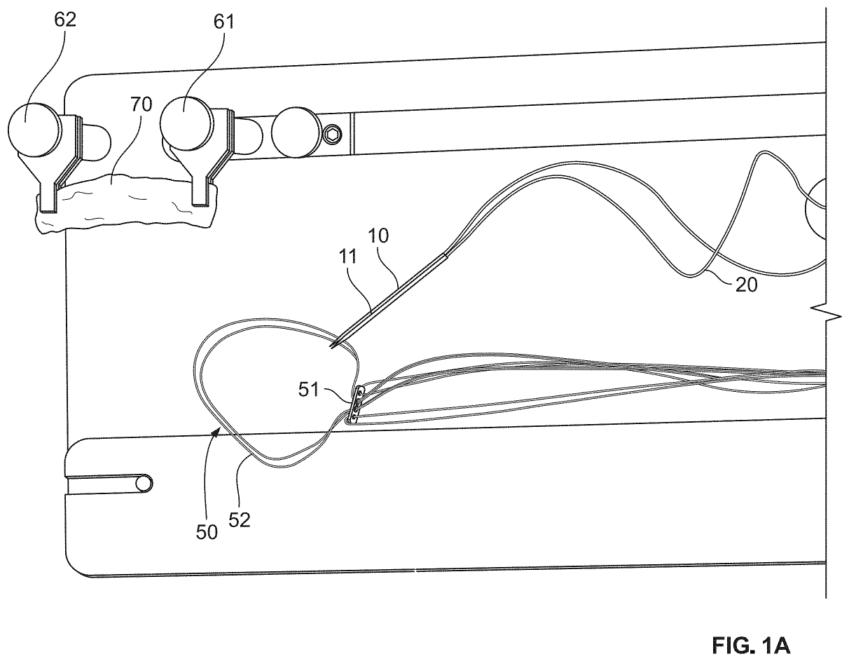

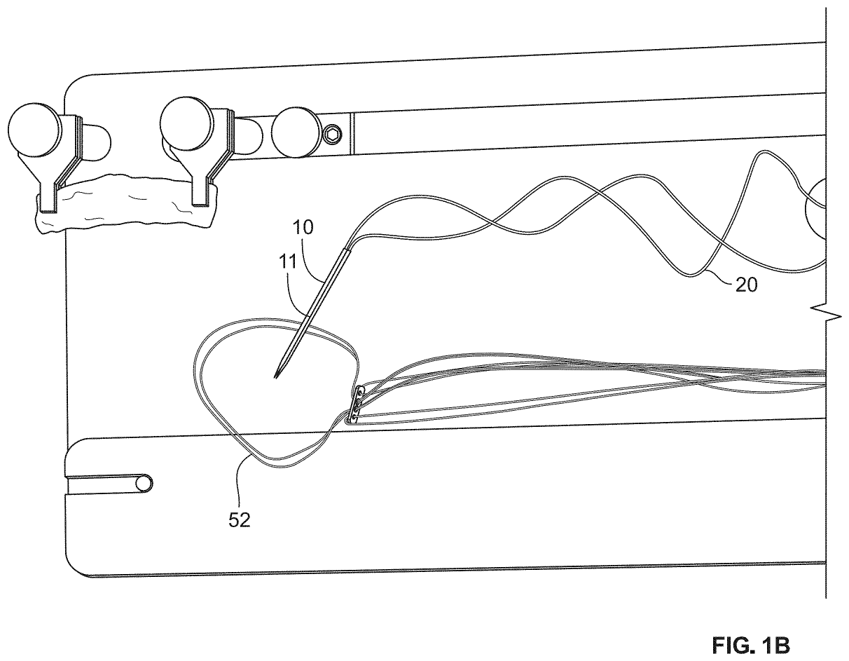

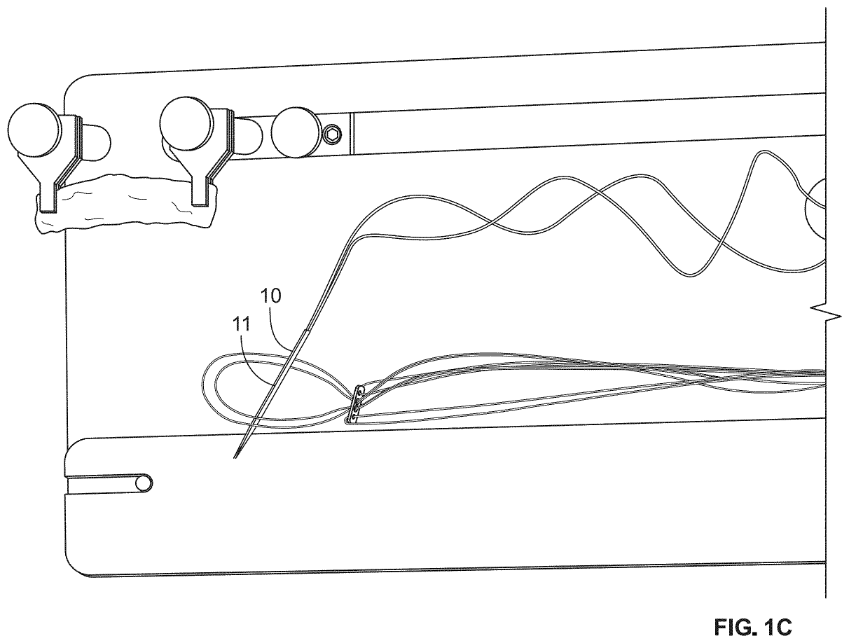

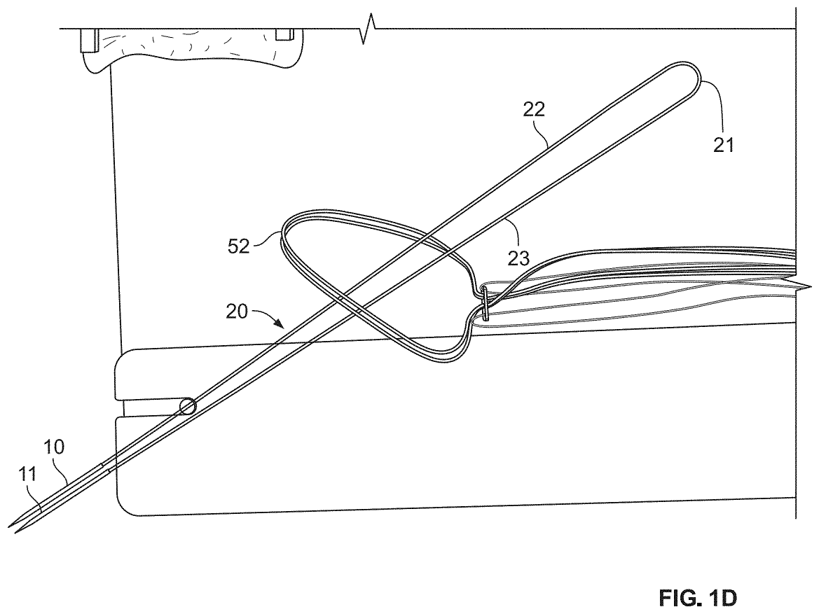

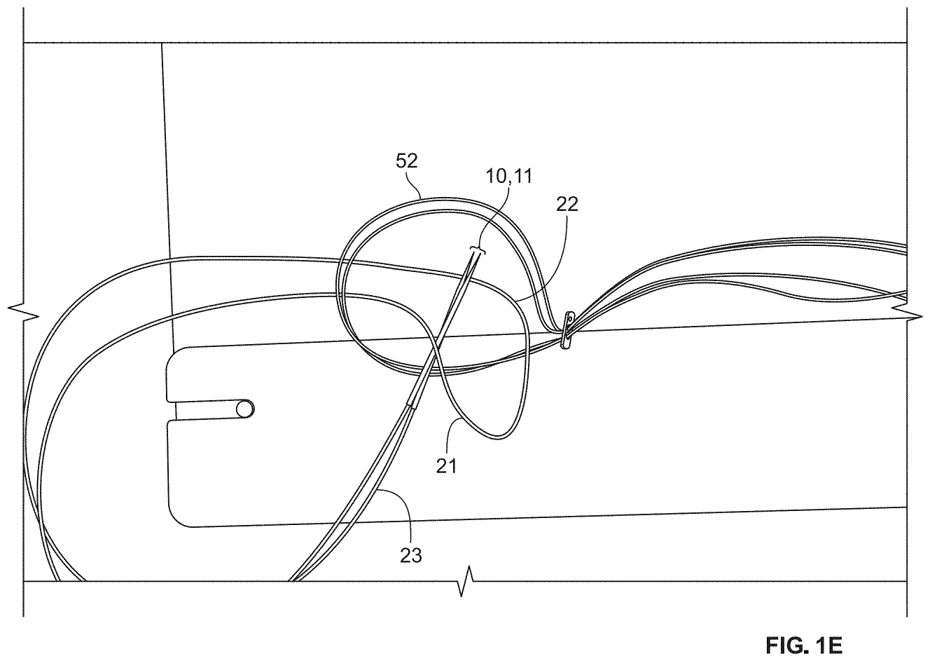

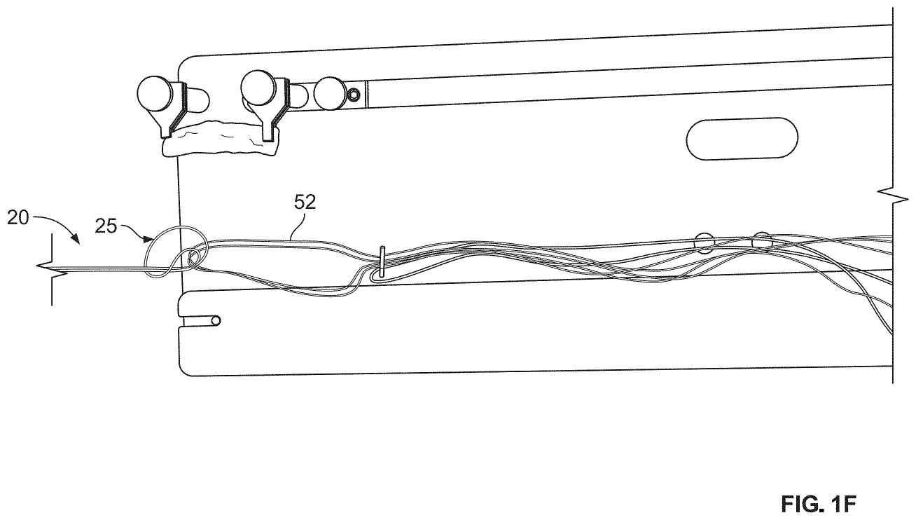

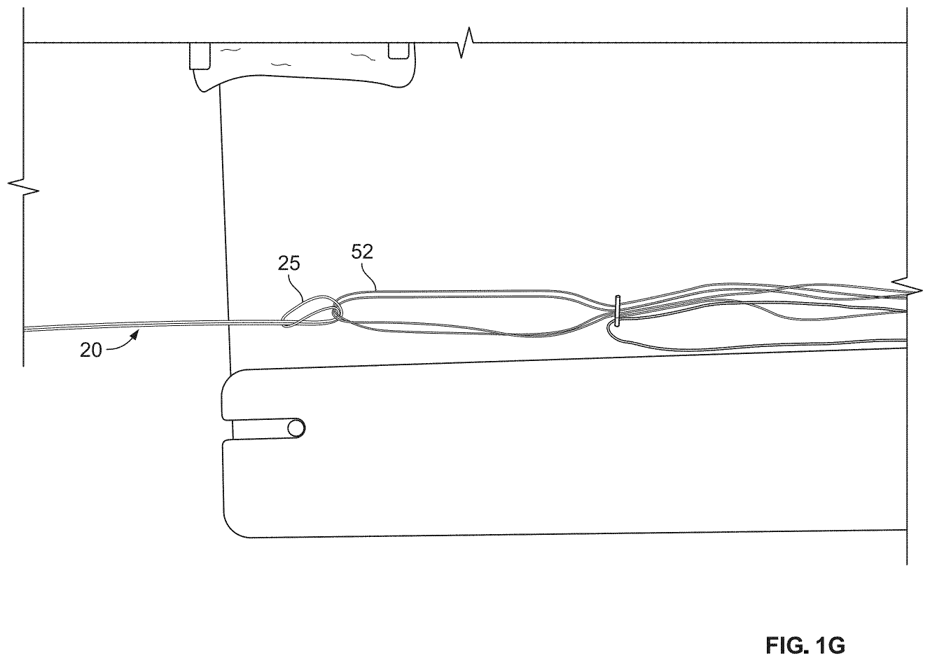

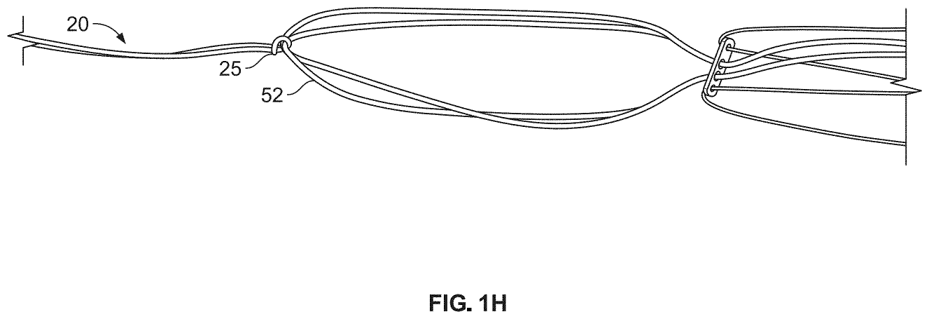

With reference to FIG. 1A, one embodiment of the present disclosure includes an implant system that can be used with a soft tissue graft to couple the soft tissue graft to a bone. The system includes a length of filament 20 with at least one needle 10 and a graft support filament 50. The support filament 50 may, as illustrated in FIG. 1A, be a suspension device used to connect and/or suspend the soft tissue graft to an anchoring site, whether a suture anchor, bone, other soft tissue, or the like. In one example, as illustrated throughout this disclosure, the support filament 50 can be a filament structure including at least one splice to form an adjustable loop 52, or filamentary loop, as is well known in the art. Support filament 50 may also include a fixation device 51, such as a button implant or all-suture suture anchor or the like, each as commonly known in the art, which can serve as the anchoring site (e.g., as known in the art, the button implant can engage a lateral cortex of the femur and/or tibia to connect and suspend an ACL graft within a tunnel formed in the femur and/or tibia). Some configurations of fixation devices 51 can be attached to the support filament 50 after the support filament 50 is passed through anatomical structures or tunnels. Buttons having open slots or enclosed holes into which the support filament 50 could be fed are examples of such fixation devices 51. The ProCinch™ family of suture suspension systems (Stryker Corporation, Greenwood Village, Colo.) offers other examples of support filaments 50 and fixation devices 51. While a graft support filament 50 including at least one adjustable loop will be discussed for illustration of the various exemplary embodiments herein, the support filament may be various different structures as known in the art for use in manipulating, securing or engaging a soft tissue graft, including a simple length of filament (such as suture) or multiple lengths of filaments, a continuous loop of filament (such as a continuous suture loop as known in the art), at least one filament including at least one knot, such as a sliding knot or slip knot, or the like.

The length of filament 20 can be a length of suture, such as a braided suture composed of more than one filament, and the suture may be either a round suture, a flat suture, commonly referred to as suture tape, or the like or any combination of shapes, colors or patterns, materials, or the like. Further, the filament can include a core or be coreless, as known in the art, and may be permanent, biodegradable, bioresorbable, or biologic, as known in the art. Also, the filament can be coated with a bone friendly substance on/in at least a portion of the filament that is anticipated to be in contact with a bone wall of a bone tunnel. Still further, the suture can be any size suture desired, and may be selected for proper strength, flexibility and elasticity for a particular application. Needle 10 may be connected to an end of filament 20. A second needle 11 can be connected to the other end of filament 20 (FIG. 1B). For example, filament 20 may be XBraid® TT, 1.4 mm, White/Blue, Suture Tape, Double Armed ST-1 (double Straight Taper Needle, 60 mm), 40″ Suture (Stryker Corporation, Greenwood Village, Colo.). This XBraid® TT product is the exemplary suture construct used in the illustrated embodiments of FIGS. 1-12. As explained in various embodiments herein, filament 20 can be used in various suturing methods to associate the support filament 50 with a soft tissue graft. Namely, needle(s) 10, 11 can be used to pass filament 20 through the soft tissue graft and around support filament 50 to couple support filament 50 to the soft tissue graft using filament 20. Thus, the term “filament” means a flexible length of material, such as a suture, that can be a monofilament or may be formed of more than one filament twisted, braided or otherwise grouped together; one example of a filament is a typical coreless or cored braided suture formed of at least ultra-high molecular weight polyethylene as commonly used in orthopedic surgery applications. The term “filamentary” relates to a structure comprising or formed of a filament; as noted above, one example of a filamentary structure or device is a braided suture formed into a construct of at least one splice in its body to create at least one adjustable suture loop.

In certain applications, such as in the repair of an ACL, via replacement/reconstruction or via removal of the remaining soft tissue and replacement with a graft, the aforementioned implant system can suspend the soft tissue graft relative to the femur and/or tibia. Typically, for instance in such an ACL reconstruction, the graft support filament 50 is associated with the soft tissue graft such that at least portions of both the graft and support filament 50 are positioned within a bone tunnel in one of the femur or tibia and fixation device 51 is positioned against the outer cortex of the particular bone through which support filament 50 is positioned. The soft tissue graft thus extends through the particular bone tunnel (in the femur or tibia), across the joint space, and into the other of the femoral or tibial bone tunnels. The soft tissue graft is then secured relative to the other bone as well, thereby suspending the soft tissue graft across the joint and between the femur and tibia.

Such systems can also be, in some embodiments, a kit. For example, a kit can include at least one graft support filament and at least one filament. Such a kit may also include a fixation device. Such a kit may also include a soft tissue graft, typically an allograft, xenograft or artificial graft. In such a kit including the soft tissue graft, the support filament and filament can already be secured to the graft, or alternatively, the various elements of the kit can be separate to be put together by the operator as desired.

Still further, if the various elements of the kit are supplied separate to be constructed by the operator (and/or packaged separately), the kit may also include step-by-step instructions, a surgical protocol, or the like, to provide guidance to an operator on how the elements may be put together, including how the graft support filament and filament may be combined with a soft tissue graft, such as an autograft. Such step-by-step instructions (or the like) may also include one or more images illustrating how the elements of the kit can be combined and used. Still further, packaging enclosing one or more of the elements can include features to help an operator combine one or more of the elements, such as paper cleats, projections, holes, or the like.

FIGS. 1A-6C illustrate one exemplary embodiment of a method of preparing a soft tissue graft. In this example, a fixation system such as that discussed above is used, and includes the length of filament 20, associated needles 10, 11, and support filament 50.

FIG. 2 illustrates a soft tissue graft 70 that can be used in this method, which in this embodiment is a quad tendon allograft. The graft 70 is illustrated as being secured between clamps 61, 62 of a standard graft preparation table, though of course graft 70 can be positioned as desired by the operator for preparation of graft 70. While graft 70 (and indeed, all grafts illustrated in the exemplary figures herein) is entirely soft tissue, and thus a support filament may be attached anywhere along its length, a graft capable of being prepared using the methods herein could also have a bone block or bone plug on one end. If such a bone block/plug is present, then such a graft could still be prepared using the methods of preparation herein as to the soft tissue portion of such a graft.

FIGS. 1A-1H illustrate an optional step that can be performed at the outset of this method, whereby filament 20 is initially associated with support filament 50. Specifically, filament 20 may be connected to the at least one adjustable loop 52 such that they can be manipulated as a unitary construct relative to graft 70. As illustrated, filament 20 is manipulated into a “luggage-tag configuration” by folding filament 20 on itself (e.g., folding filament 20 in half) such that the ends of the filament 20 are together at one end and a loop is formed at the other end. Both ends of the filament (and associated needles 10, 11 if present) are then passed through the at least one adjustable loop 52 (FIGS. 1A-D), passed around the at least one loop 52, and then passed in between the two lengths 22, 23 of filament 20 between loop 52 and the formed loop 21 of filament 20 (FIG. 1D-1E). The ends are then pulled tight, resulting in a luggage-tag configuration 25, also known as a lark's head knot or cow hitch (FIGS. 1F-1H). Other techniques to associate filament 20 and support filament 50 may also be used, such as a half hitch, a clove hitch, or other such knot, or even simply passing filament 20 one or more times around the at least one loop 52.

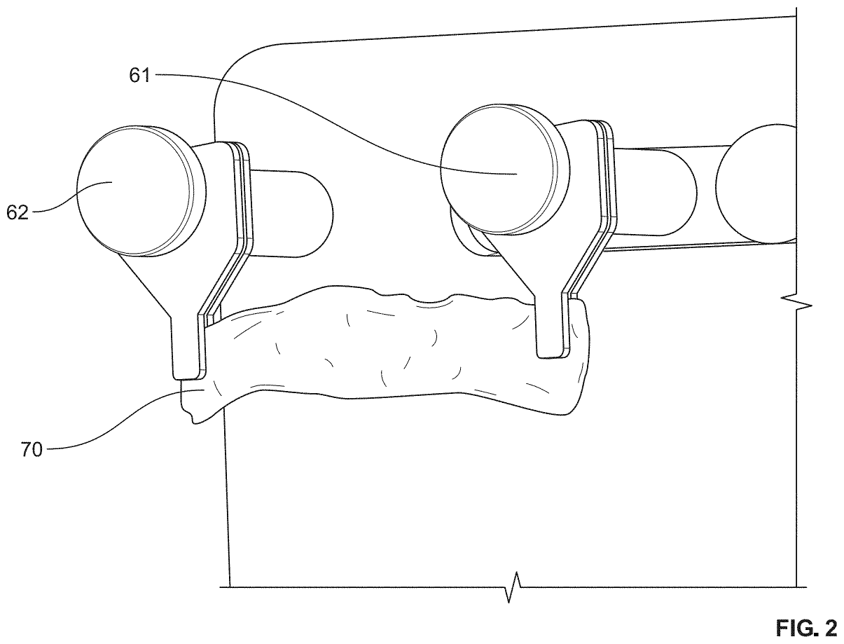

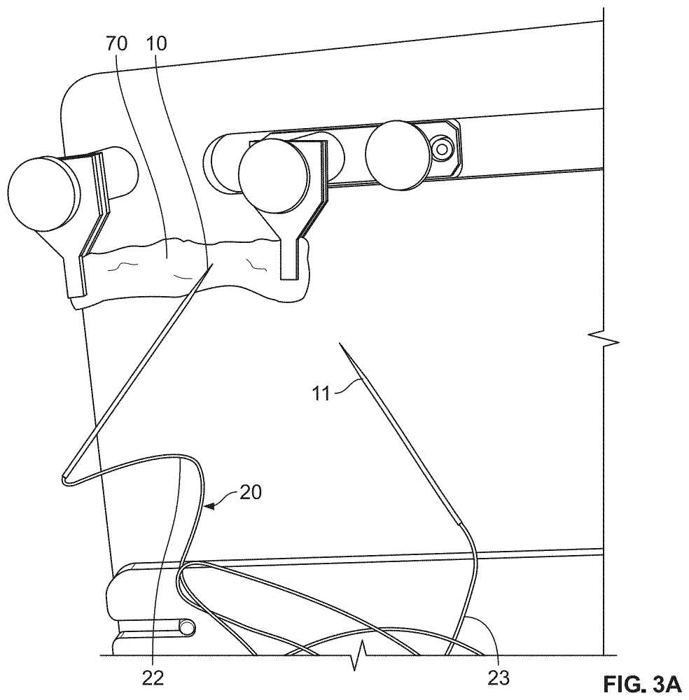

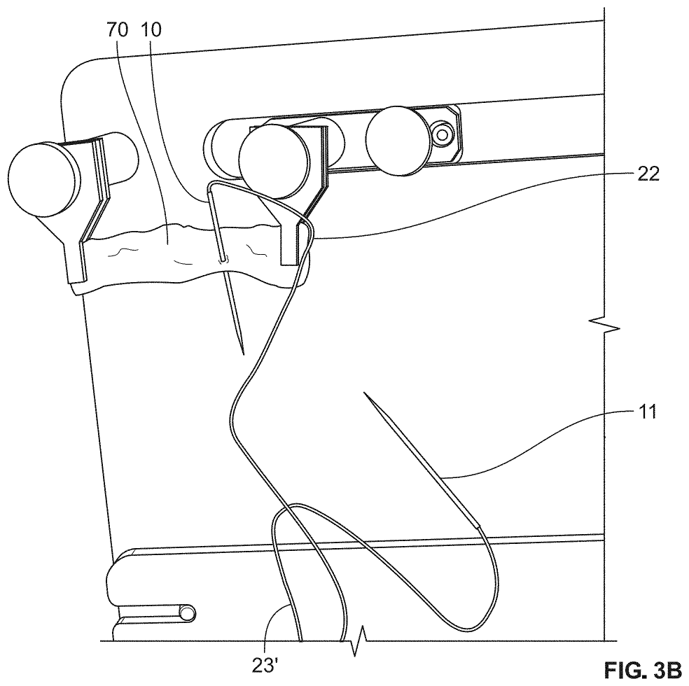

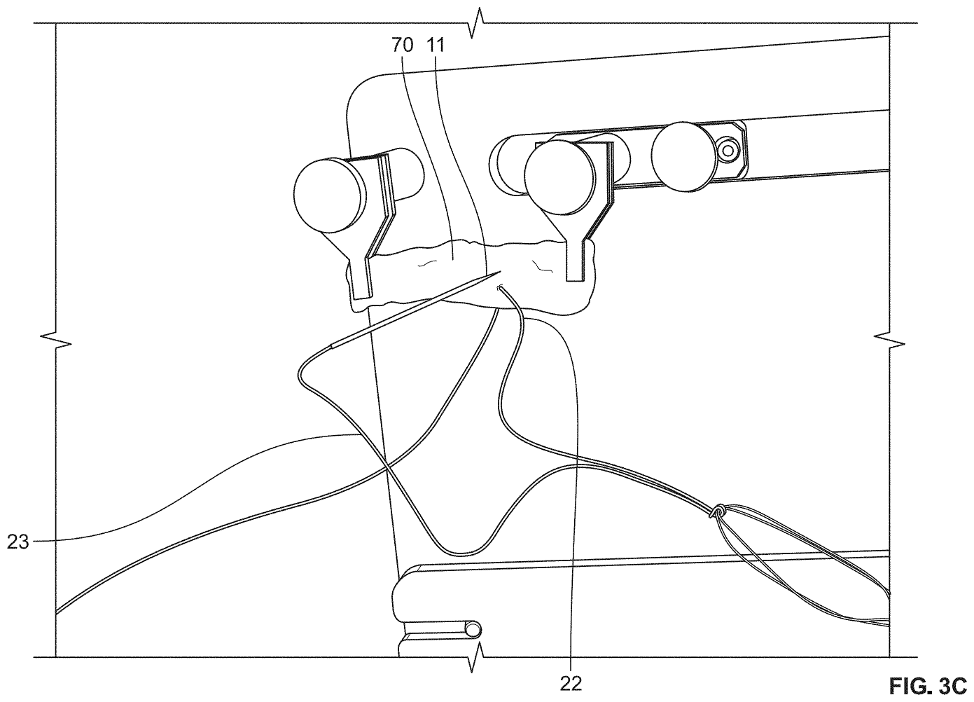

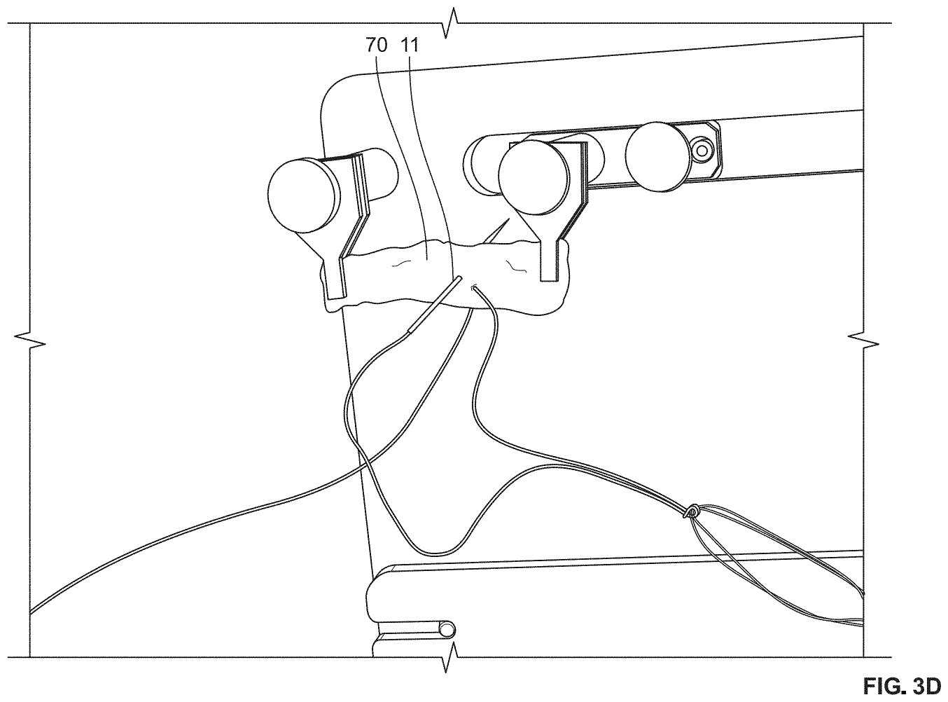

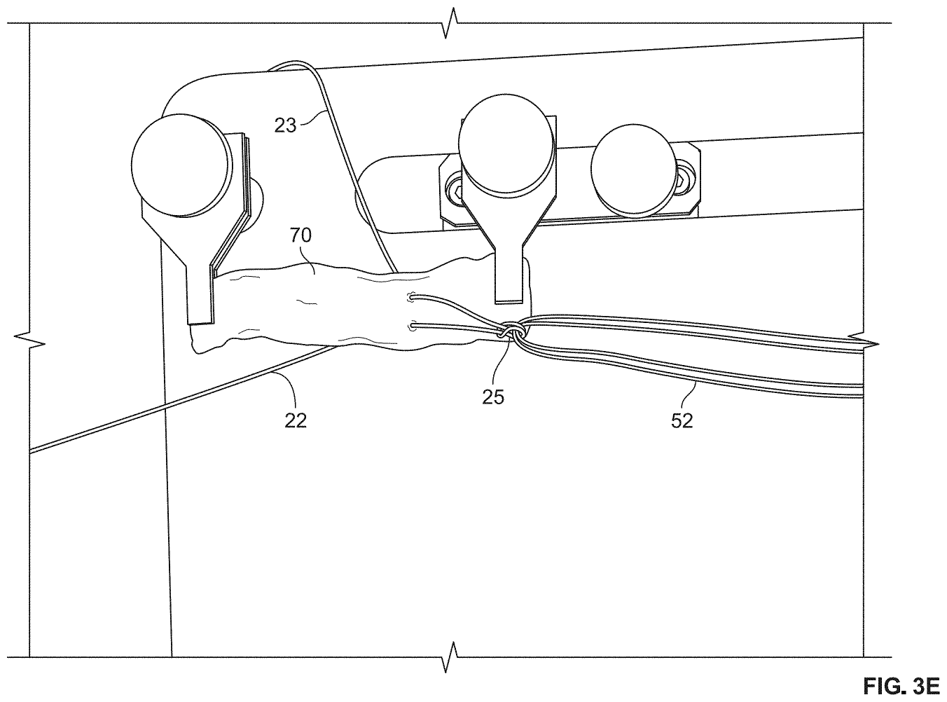

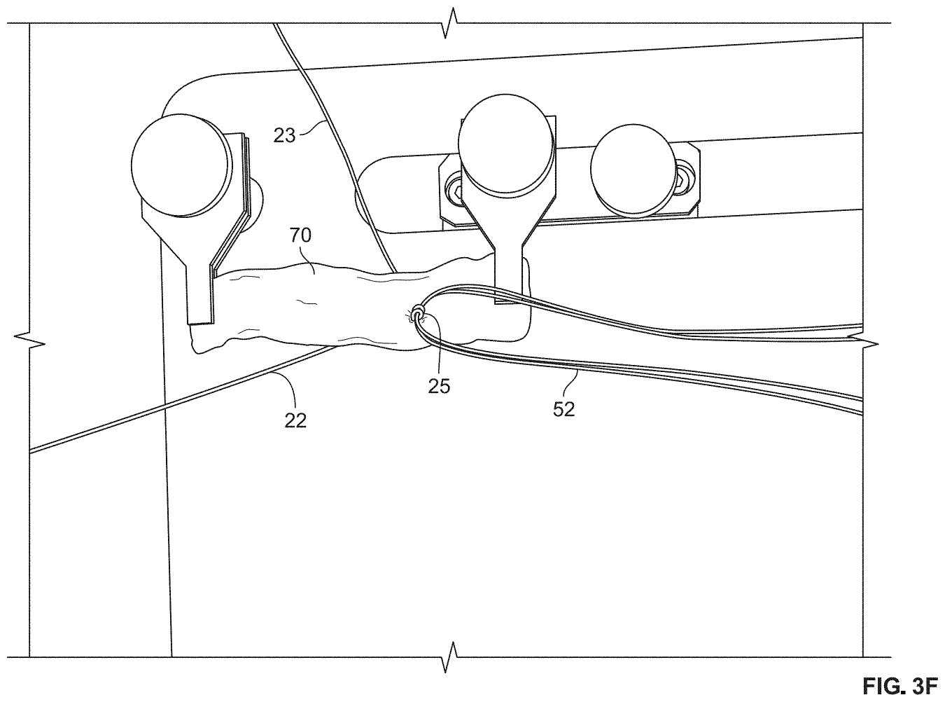

Continuing with this exemplary method, FIGS. 3A-3F illustrate that needles 10, 11 are each passed through graft 70 (FIGS. 3A-3D). The associated filament lengths 22, 23, respectively, of filament 20 ends, are passed through graft 70 as needles 10, 11 are pulled. As illustrated, needles 10, 11 are passed relatively close to one another (e.g., in this example, about 2 cm from respective ends of the graft 70 and about 2-5 mm from one another and about 2-5 mm from the sides of the graft) but they may be positioned anywhere along the graft as desired including both needles passing through the same first position on the graft. The illustrated positioning, or within a few centimeters of this location, may be useful to position most of the at least one adjustable loop 52 along the length of graft 70, thereby ensuring a secure and strong connection between loop(s) 52 and graft 70 and to ensure a close position of fixation device 51 and the end of graft 70. Once through the graft, needles 10, 11 and lengths 22, 23 are further pulled to associate support filament 50 relative to graft (FIGS. 3E-3F). For instance, FIG. 3F shows the luggage-tag configuration 25 abutting a top surface of graft 70 and filament 20 extending from the luggage-tag configuration, through graft 70, and exiting out a bottom surface of graft 70. As such, support filament 50 is brought into relation with graft 70, such as is illustrated, support filament 50 can abut graft 70 along with luggage-tag configuration 25. As an alternative embodiment, after lengths 22, 23 are passed through graft 70 they can be tied together to form a knot on the undersurface of the graft in an effort to create a more static fixation point.

As noted above, if the optional steps of FIGS. 1A-1H were not performed (or another variation to associate filament 20 and support filament 50 was not performed), filament 20 and support filament 50 would remain separate and manipulated relative to graft 70 as separate structures. As such, for example, one of the lengths 22, 23 of filament 20 may simply be passed through the at least one adjustable loop 52 of support filament 50 prior to needles 10, 11 passing through graft 70 such that, once the lengths 22, 23 of filament 20 are tensioned through graft 70, support filament 50 may be brought into abutment with graft 70.

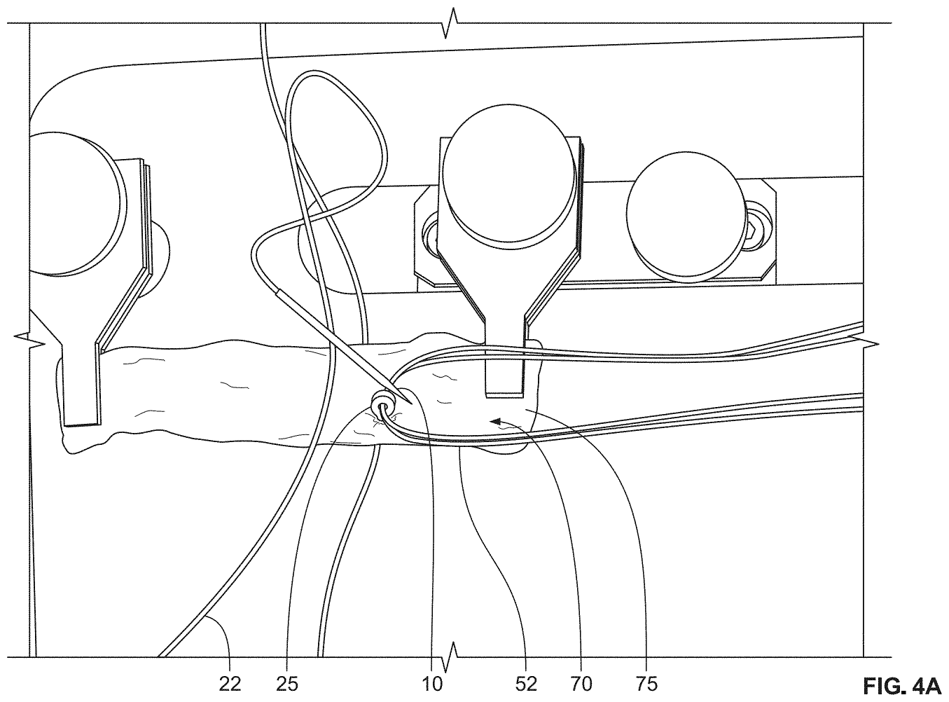

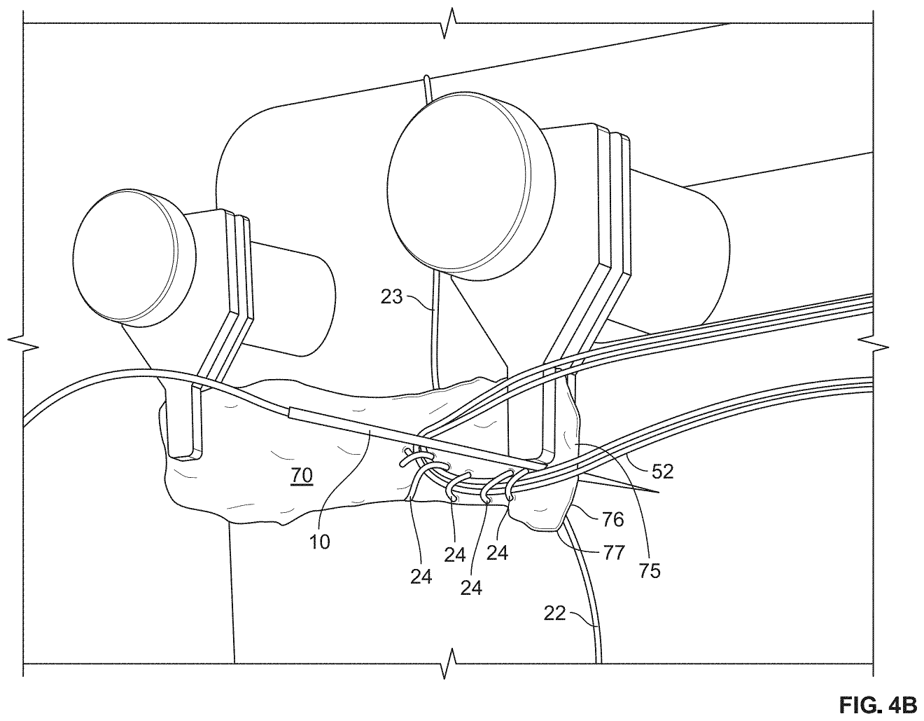

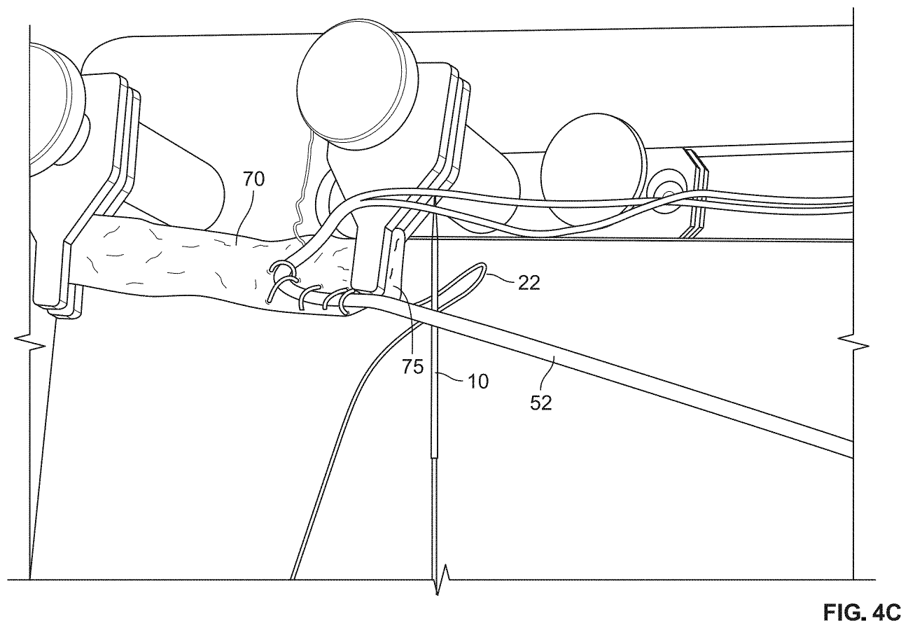

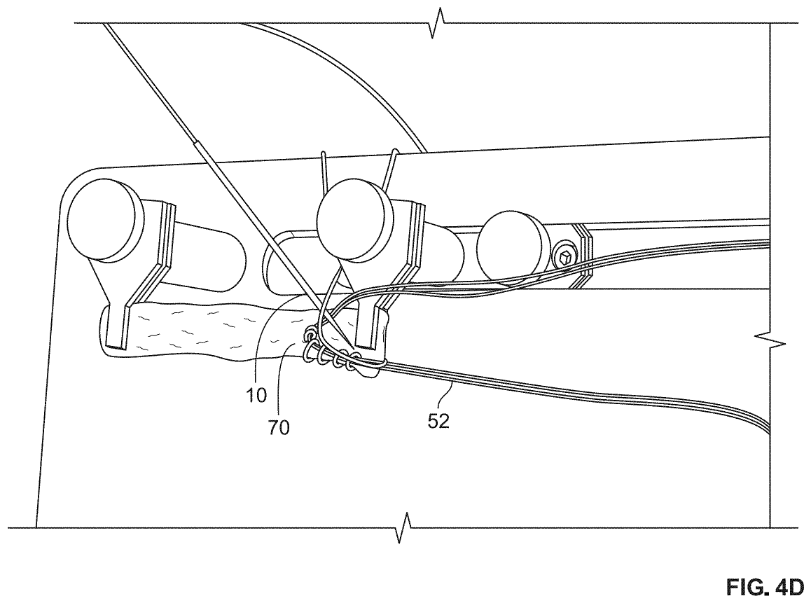

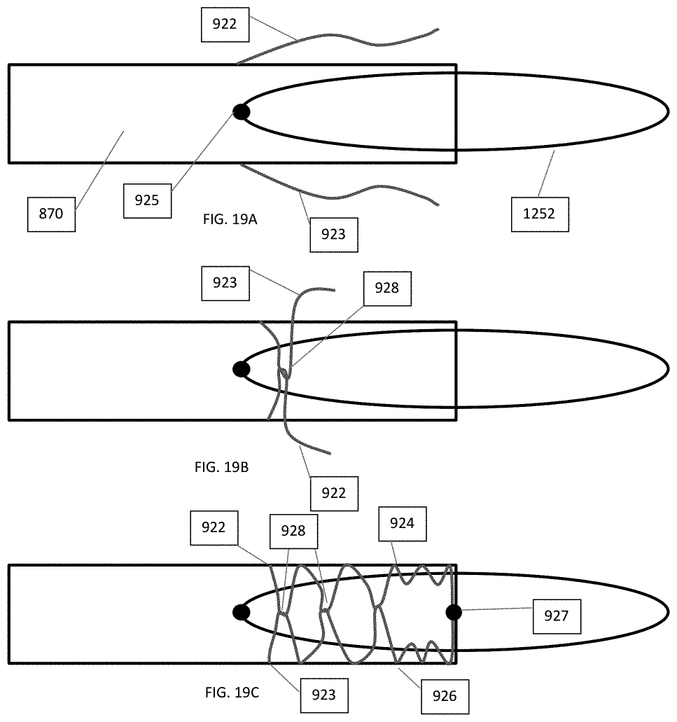

Next, as illustrated in FIG. 4A, one length 22 of filament 20, and as illustrated needle 10, is brought around the lateral edge of the graft 70, over the at least one adjustable loop 52, and passed through the top surface of graft 70, adjacent to and within the circumference of the at least one loop 52. This maneuver may be repeated one or more times as desired, forming, for example as illustrated, a running stitch pattern along the length of at least a portion of graft 70 (e.g., from the location where luggage-tag configuration 25 was brought into abutment with graft 70 and towards one longitudinal end 75 of graft 70) and along the length and around the filament of the at least one loop 52. In other words, filament 20 forms a spiral pattern along the graft 70 and around the at least one loop 52 in a first direction (FIG. 4B). As illustrated in FIG. 4B, the first direction is a counterclockwise direction along the at least one loop 52, from the luggage-tag configuration 25 location along the loop(s) 52 towards the graft end 75. For each of these passes in the spiral pattern 24 using length 22, filament 20 does not pass through the filament of the at least one loop 52, but instead passes around the filament of the at least one loop 52. Such a configuration allows for the at least one loop 52 to slide relative to graft 70 and filament 20, and in other words, maintains the functionality of the at least one loop 52 in that the size of the loop can be adjusted. While a running stitch (e.g., a continuous passing of suture) is illustrated, other suture techniques are also envisioned. For example, a locking Krackow stitch may also be used, such that, at each pass (e.g., similar to passes 24 in FIG. 4B) of the suture around the loop 52 and through graft 70, filament 20 assumes a locking feature. Such a locking stitch may contribute to the integrity and rigidity of the prepared graft 70 and fixation system. Furthermore, additional circumferential wrapping around the graft prior to any subsequent needle pass(es) is also envisioned to further ensnare and tubularize the graft (e.g., such as the technique illustrated in FIGS. 19A-C), which may also include the formation of additional knots at various points during these wrapping steps.

As length 22 of filament 20 makes passes 24 along the graft 70 and approaches the longitudinal end 75 of graft 70, the operator may include one or more passes, and as illustrated two passes (FIG. 4B), of length 22 through, the lower, top and bottom corners 76, 77 of graft 70 at end 75. These final passes 24 of length 22 through these corners may assist in forming a tapered shape in the end 75 of graft 70. Such a tapered shape can allow for easier passing of the graft 70 through a bone tunnel in a bone, and further, may assist in establishing more of a cylindrical shape in the graft which may more closely match the shape of the bone tunnel and thus provide easier entry of the graft into the tunnel as well as greater surface contact between the bone tunnel and the graft, thereby allowing a better chance of establishing tissue ingrowth between the graft 70 and surrounding bone.

Continuing with this exemplary embodiment, after passing length 22 through the corners 76, 77 at the end 75 of graft 70, length 22 (and needle 10) are passed around support filament 50, beyond the end 75 of graft 70 and through loop(s) 52 (FIG. 4C), and the needle 10 repeats the purse-string or spiral suture pattern back up the graft, away from end 75 and back along the graft 70 and around the support filament 50 towards the luggage-tag configuration 25 location (if used) (FIG. 4D).

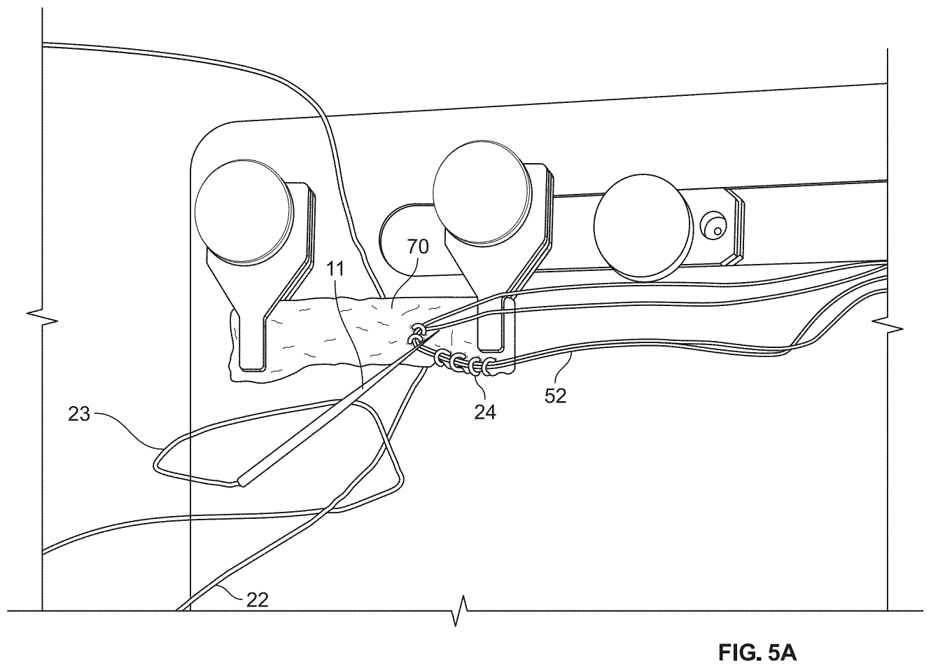

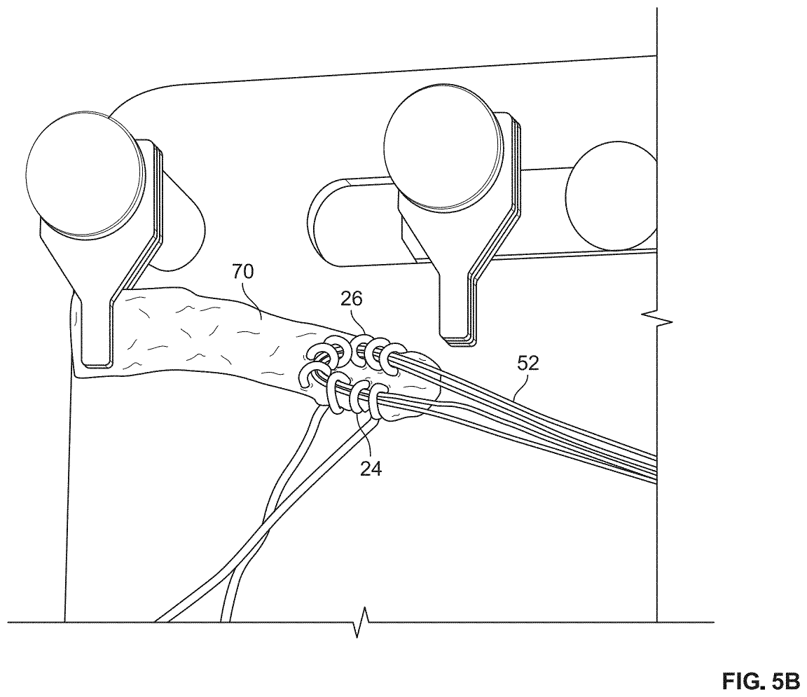

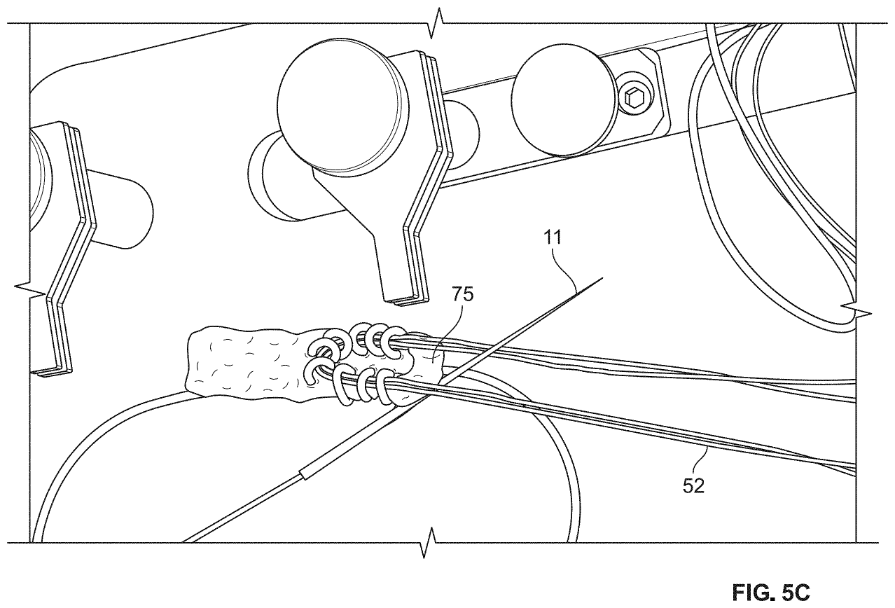

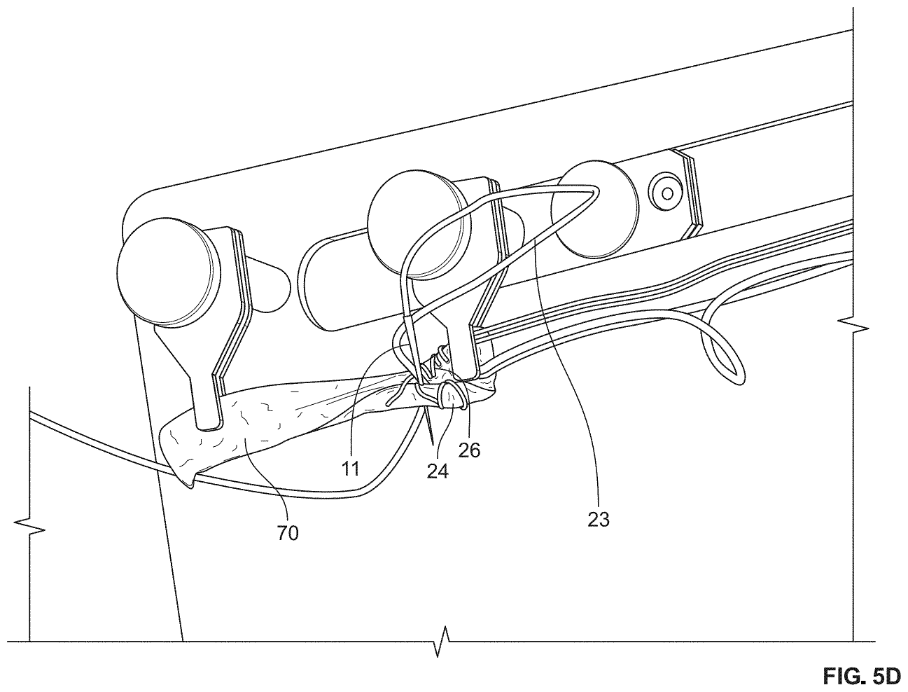

FIGS. 5A-5D illustrates the same technique as described above as to FIGS. 4A-4D, but with the other length 23 of filament 20 (and needle 11). Needle 11 and the other length 23 of filament 20 perform a similar running stitch or spiral suturing pattern, as discussed above, but, as in FIGS. 5B-5D passes 26 using length 23 are positioned along the other lateral edge of graft 70 and around the portion of the at least one adjustable loop 52 that was not ensnared by the previous suturing in FIGS. 4A-4D. Filament 23 thus travels along the graft 70 and around the at least one loop in a second direction, as illustrated, a clockwise direction along the at least one loop 52, from the luggage-tag configuration 25 location along the loop(s) 52 towards the graft end 75. FIG. 5B illustrates the result of both filament lengths 22, 23 stitching around the at least one loop 52 in opposite first and second directions, counterclockwise and clockwise respectively, to surround the loop(s) along at least part of the length of overlap of the graft 70 and the loop(s). FIG. 5D shows how graft 70, around end 75, establishes a tapered shape once filament 20 passes 24, 26 have been tensioned along graft 70 and around the at least one loop 52.

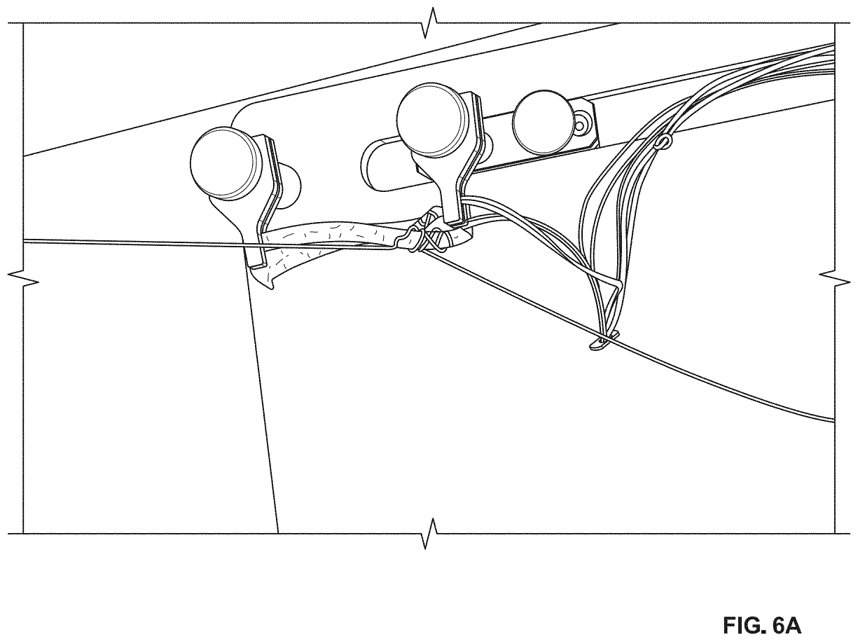

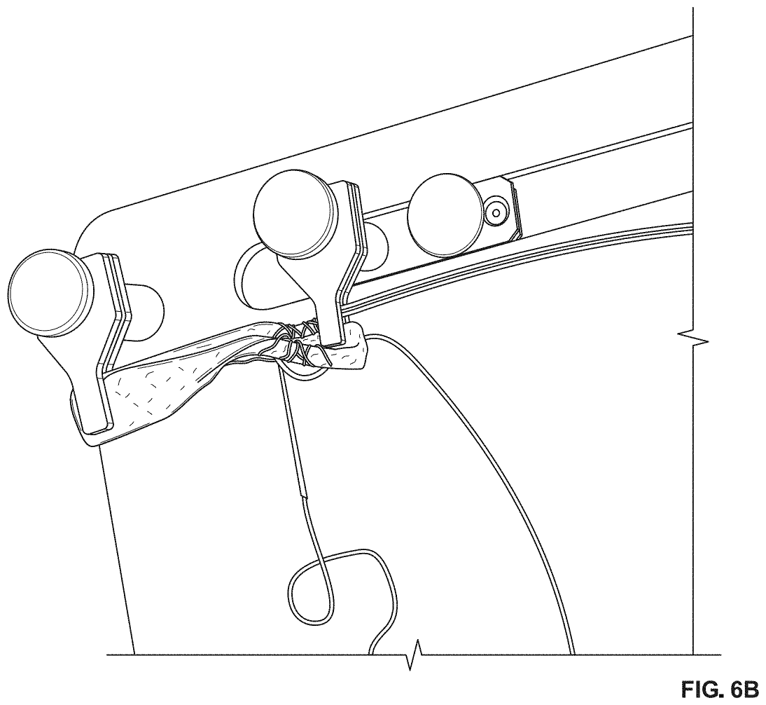

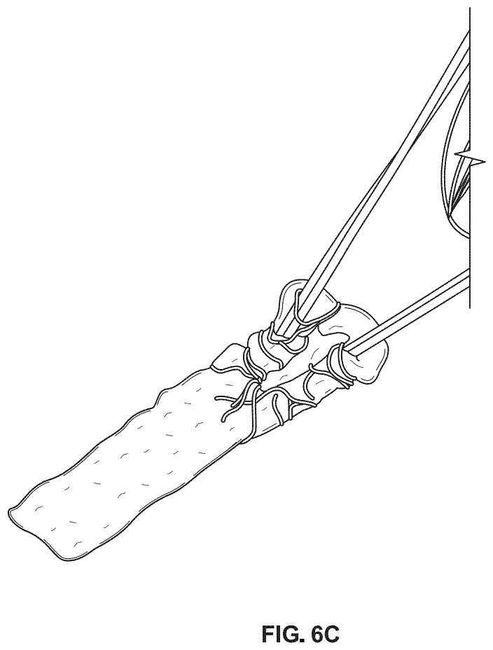

With both lengths 22, 23 of filament 20 now positioned near luggage tag configuration 25, the two lengths can be secured to one another, such as by a knot or the like (FIG. 6A). The knot can then be buried into the body of graft 70 by passing both lengths 22, 23 back through graft 70 and tensioning to pull the knot against and/or into graft 70 (FIG. 6B). Then, the tails of filament 20 can be cut at a tendon-filament interface (formed upon the filament lengths 22′, 23′ exiting from the graft after the step of burying the knot). Alternatively, the tails may remain uncut and utilized for graft passage and/or backup fixation, such as for example if being used with a traditional screw implant or interference implant, such as would be traditional in tibial fixation, the tails can be left as is and used with such an implant, or alternatively, the tails can be secured at an anchoring site, such as to the bone, to other soft tissue, to a suture anchor, or the like. More commonly the tails can be fed through holes or slots in a button fixation device and tied; again as backup fixation.

Graft can then be removed from clamps 61, 62 (if used previously) and the operator can test the at least one adjustable loop 52 to ensure it can slide relative to graft 70 and within the suture construct of filament 20. For instance, the operator can pull on either end of at least one loop 52, extending away from graft 70, to ensure loop(s) 52 can slide (FIG. 6C). FIG. 6C again also shows the tapered shape graft 70 establishes once filament 20 is tensioned and secured relative to graft 70 (e.g., knotted or otherwise).

Variations on the suturing pattern using filament 20 are also envisioned. For instance, instead of completing the suture pattern with the first length 22 of filament 20 and then repeating the same steps with the second length 23 of filament 20, the suture ends can be used in alternating fashion to eventually arrive at the position for final securement such as applying a knot as in FIG. 6A. For instance, first length 22 can be used until arriving at the end of graft 70, as in FIG. 4B-C, and then length 23 can be used to also arrive at the end of graft 70 as in FIG. 5C. Then, the return suturing of the length 22 and length 23 (in either order) can be performed to eventually arrive at FIG. 6A. In yet another alternative, each end of filament 20 can be passed around loops 52 and through tissue 70 in an alternating pattern, such that the operator switches between length 22 and length 23 with each respective pass 24, 26 through graft 70 and around loop(s) 52.

Still further, at various points during the suturing steps of the previously disclosed embodiment, the operator may wish to apply tension to filament 20 to ensure each pass 24, 26 of filament 20 around loop(s) 52 and through graft 70 is individually tensioned. While this is likely necessary if a locking stitch (e.g., locking Krackow stitch) is used, this intermittent tensioning may also be useful when using the continuous stitch as illustrated in FIGS. 1A-6C. As such, intermittent tensioning of filament 20 may occur after each pass 24, 26 of filament 20 through graft 70, after every other pass 24, 26 of filament 20 through graft 70, after every n-th pass 24, 26 of filament 20 through graft 70, upon filament 20 being passed through the end 75 and/or corners of graft 70, upon filament 20 returning back to the luggage-tag configuration 25 location, just before the filament 20 is secured (as in FIG. 6A), and/or any combination thereof.

Such intermittent tensioning may be useful in other suturing patterns as well. For instance, instead of ends 22, 23 forming passes 24, 26, respectively, in a linear fashion along one side of the graft, they may alternate from one side of the graft to the other with each pass. Such suturing may form a “shoelace” style pattern in which ends 22, 23 continuously pass back and forth from one side of the tendon to the other. Since the suture ends 22, 23 may experience added friction due to the intermittent passing across the width of the graft (and around differing portion of the loop(s) 52), intermittent tensioning can be useful to help ensure consistent tension on each pass 24, 26. This suture pattern may also provide additional tensioning to further impart a tapered or tubular shape to the graft 70.

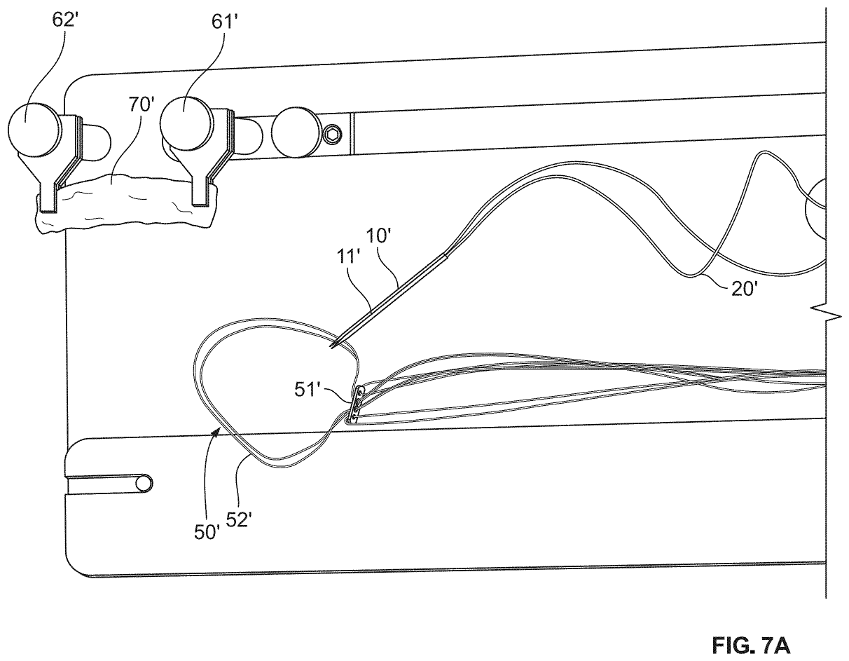

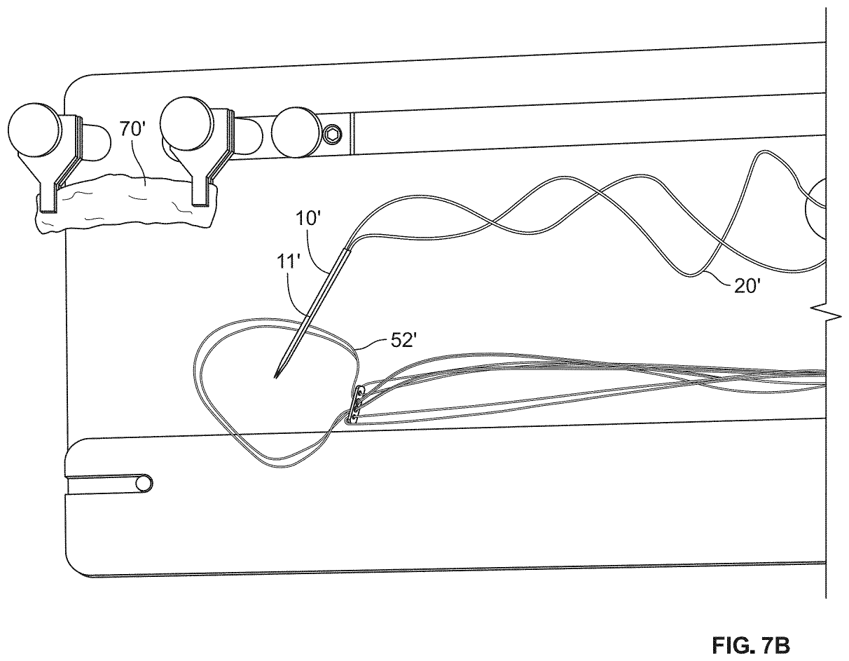

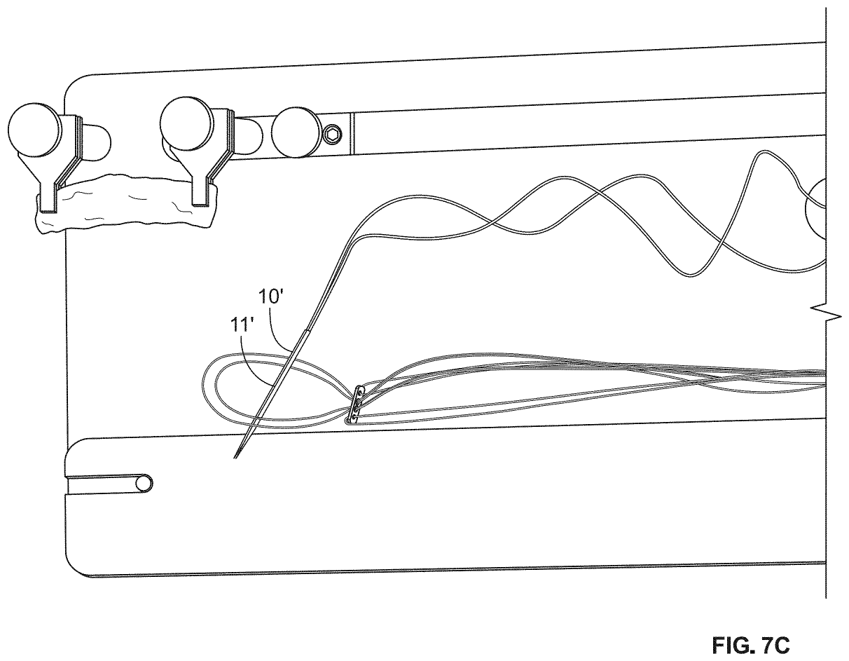

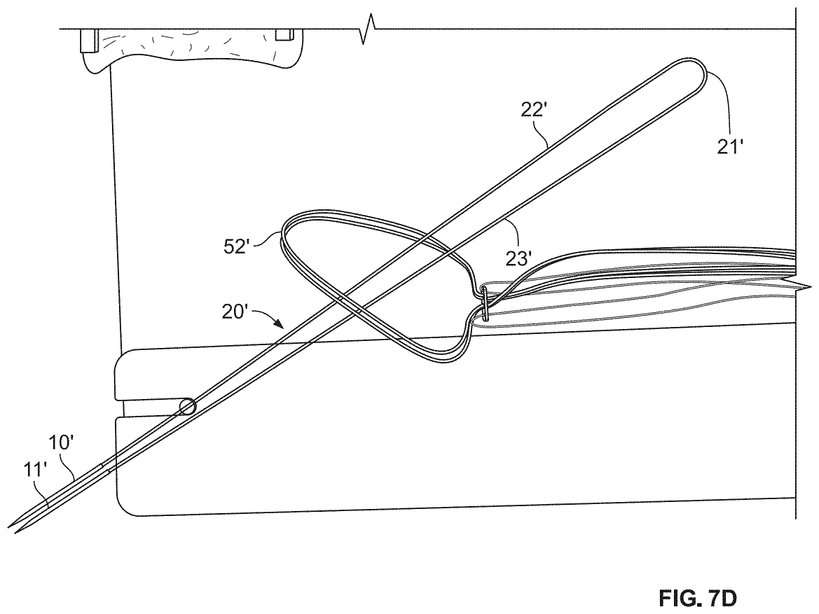

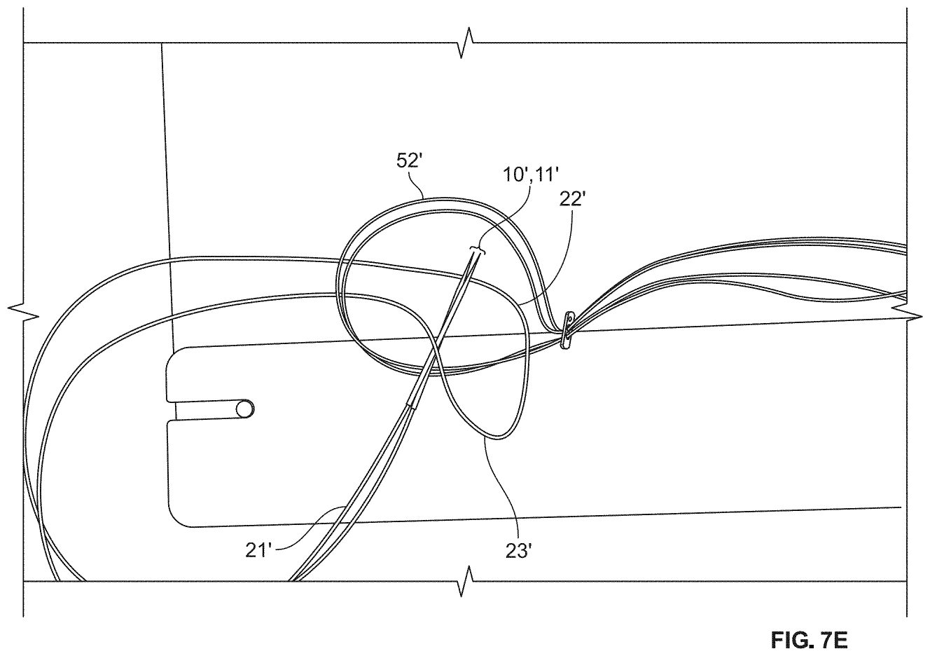

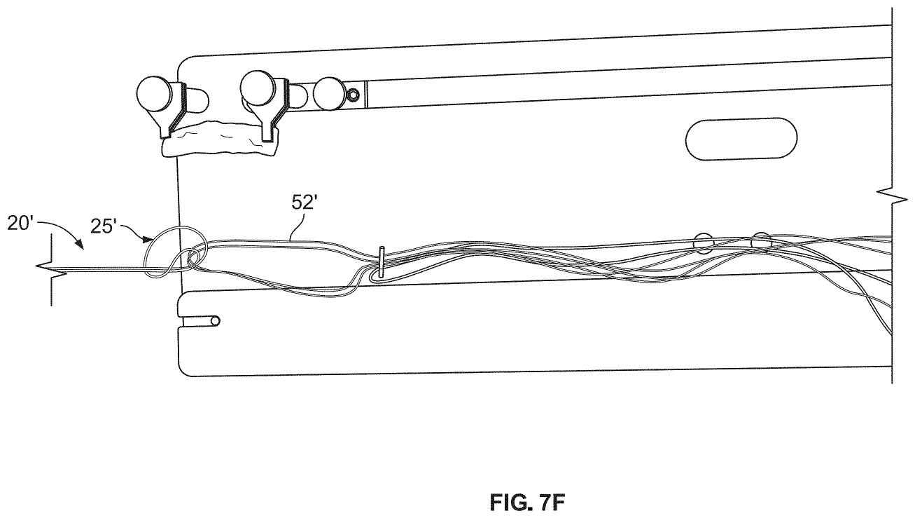

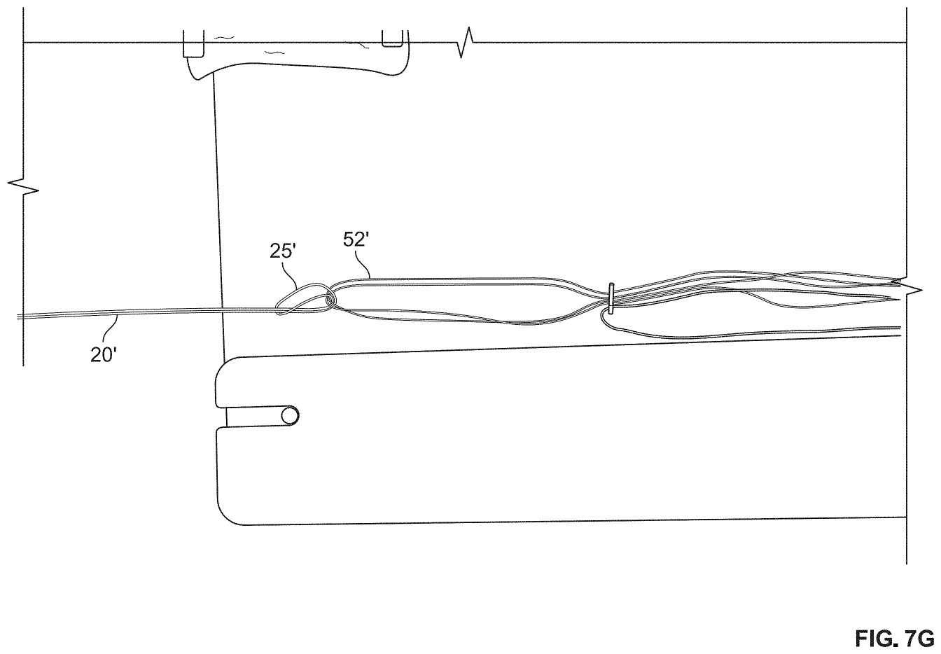

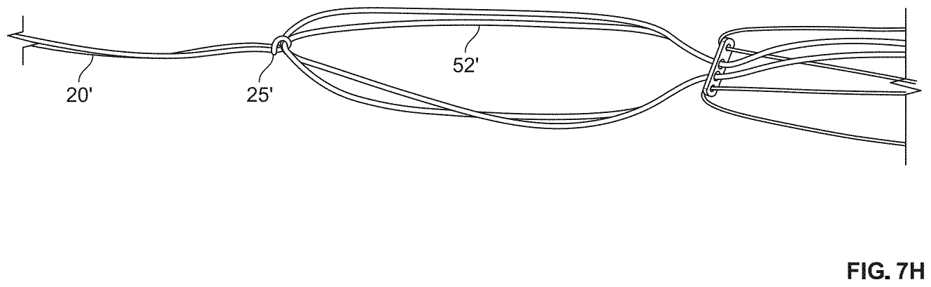

FIGS. 7A-12E illustrate another exemplary embodiment of a method of preparing a soft tissue graft, again utilizing as an example a fixation system as that described above with reference to FIGS. 1A and 1B, though in this instance described using similar reference numbers including a length of filament 20′, associated needles 10′, 11′, and graft support filament 50′. Similarities between reference numerals in FIGS. 7A-12E to those above in FIGS. 1A-6C are intended to show similarity in structure and use, and thus descriptions above are equally applicable to this embodiment, with alternative and/or additional description set forth as follows.

In this embodiment, FIGS. 7A-7H and 8 illustrate the same method steps and devices as illustrated and discussed above in the previous embodiment in FIGS. 1A-1H and 2, and will not be repeated here. Though it should be noted that, as per the illustrated reference numbers in this example, the implant system includes a length of filament 20′ with at least one needle 10′ and a graft support filament 50′ which, as described above, can be formed into a suspension device including at least one adjustable loop 52′ and a fixation device 51′. Also similar to the previous embodiment, the length of filament 20′ can be manipulated into a “luggage-tag configuration” 25′ or the like to associate filament 20′ and support filament 50′ (FIGS. 7A-7H).

FIG. 8 illustrates a soft tissue graft 70′ similar to soft tissue graft 70 of FIG. 2, again illustrated as secured between claims 61′, 62′.

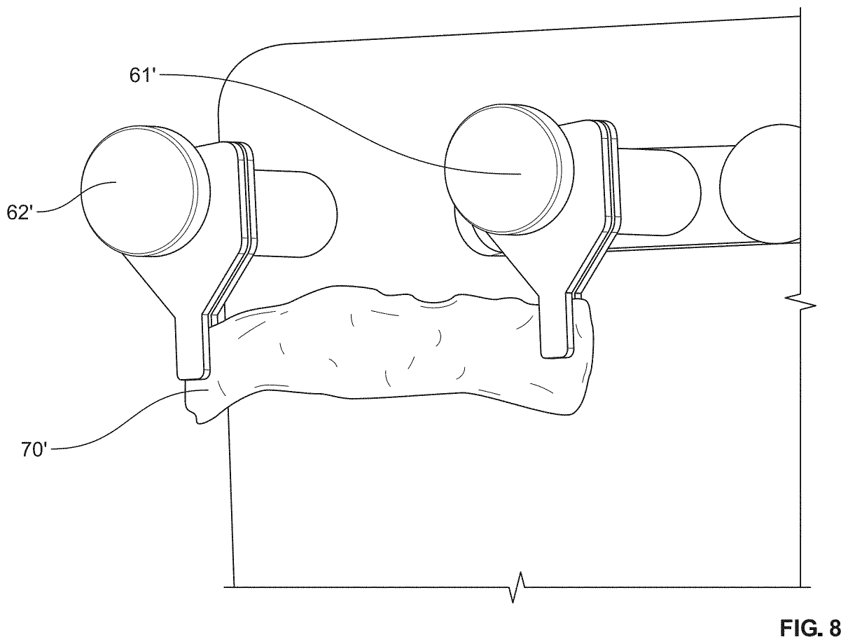

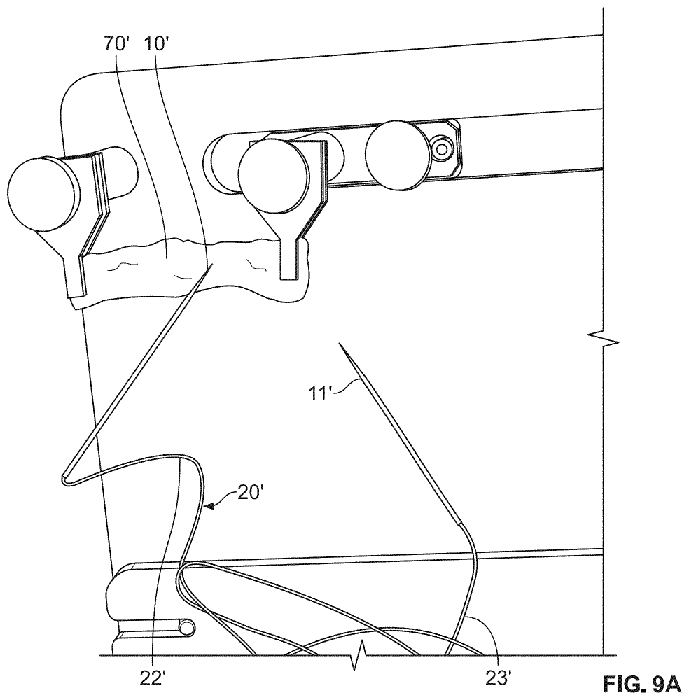

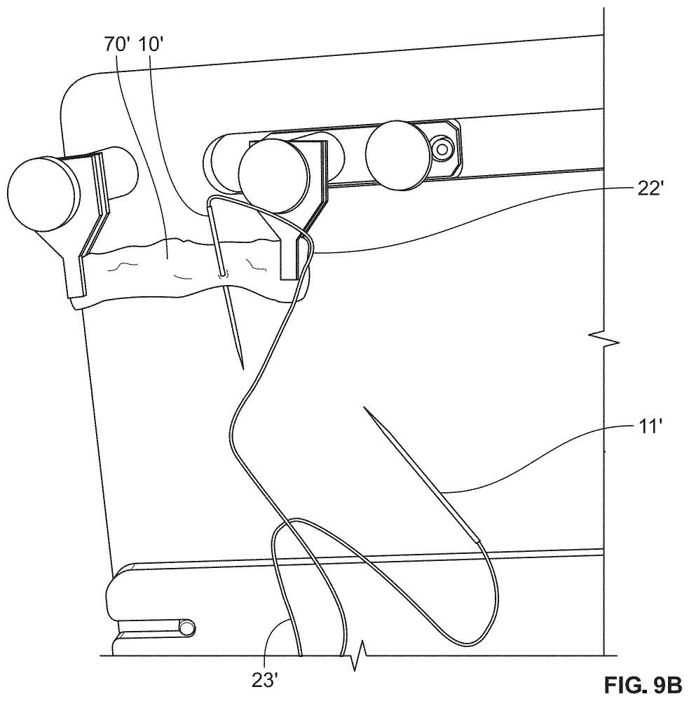

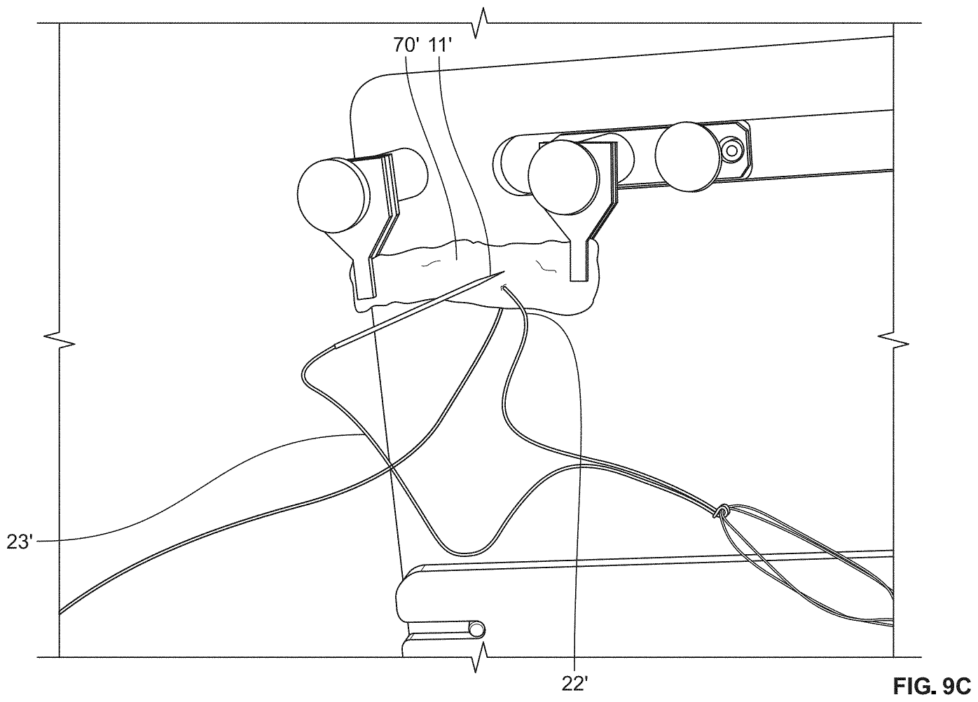

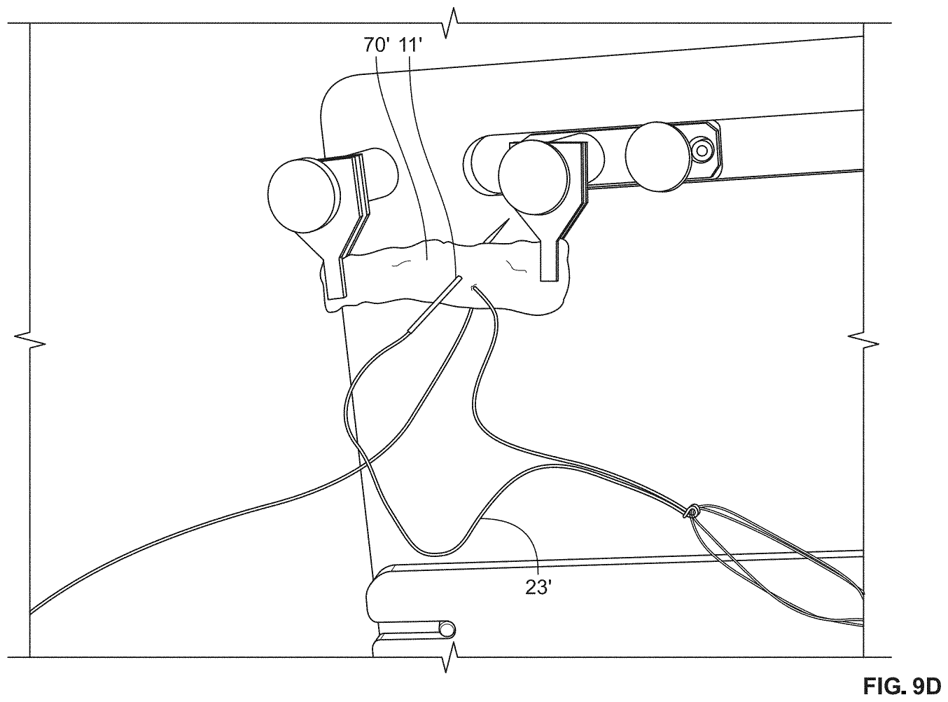

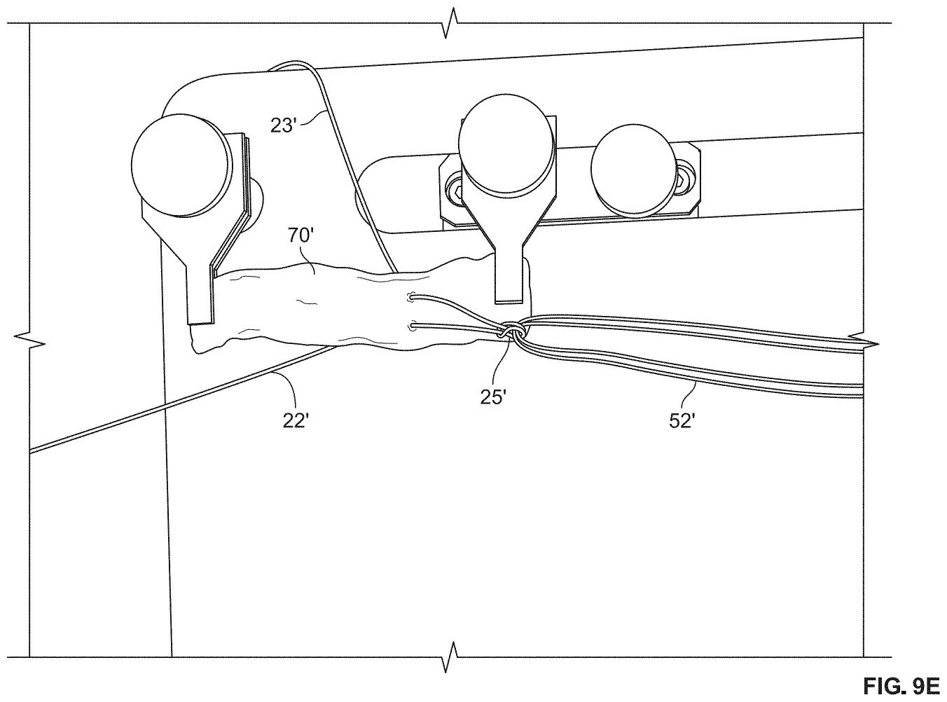

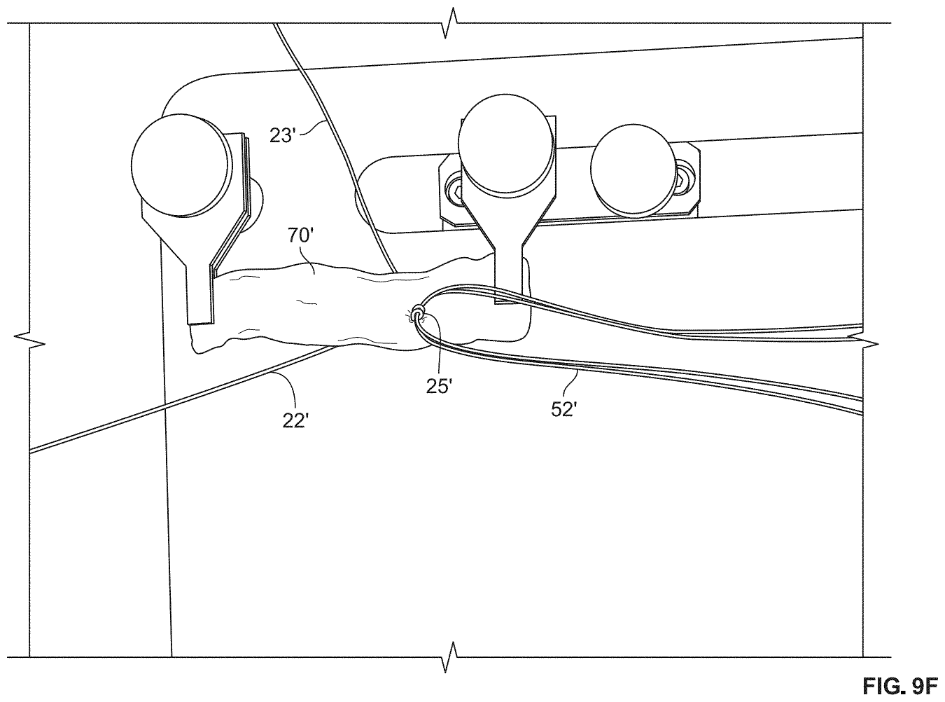

Continuing with this embodiment, FIGS. 9A-9F are again similar to FIGS. 3A-3F, discussed above to position luggage-tag configuration 25′ into abutment with a top surface of soft tissue graft 70′ and filament 20′ extending through and out a bottom surface of graft 70′. Further, the above discussion of FIGS. 1A-1H being optional applies equally to FIGS. 7A-7H, as do the alternative ways to associate the filament 20′, graft 70′ and fixation device 50′.

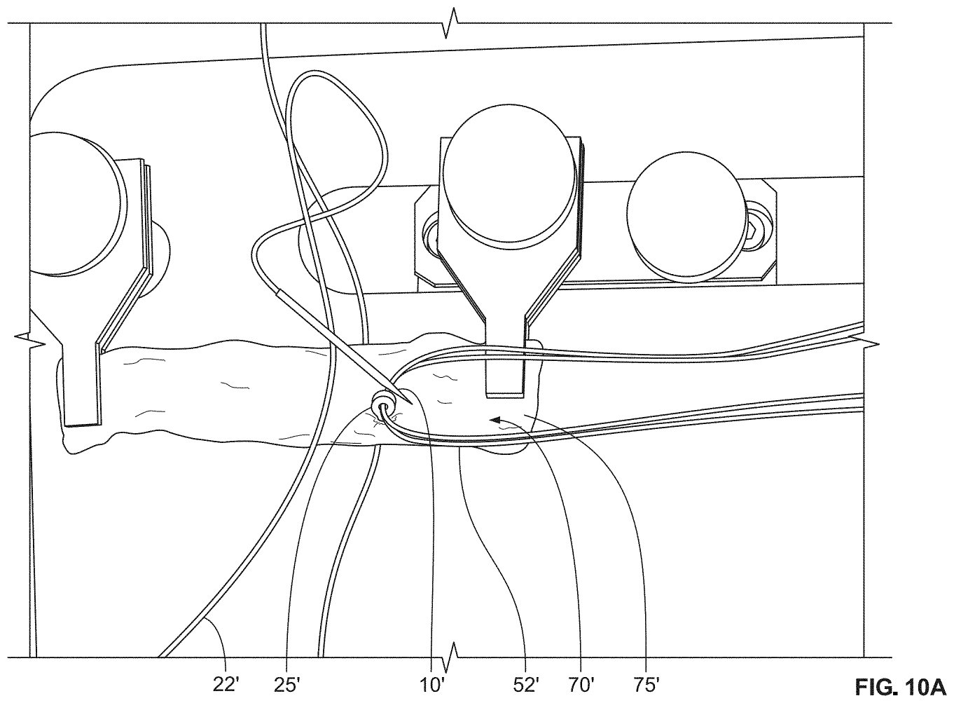

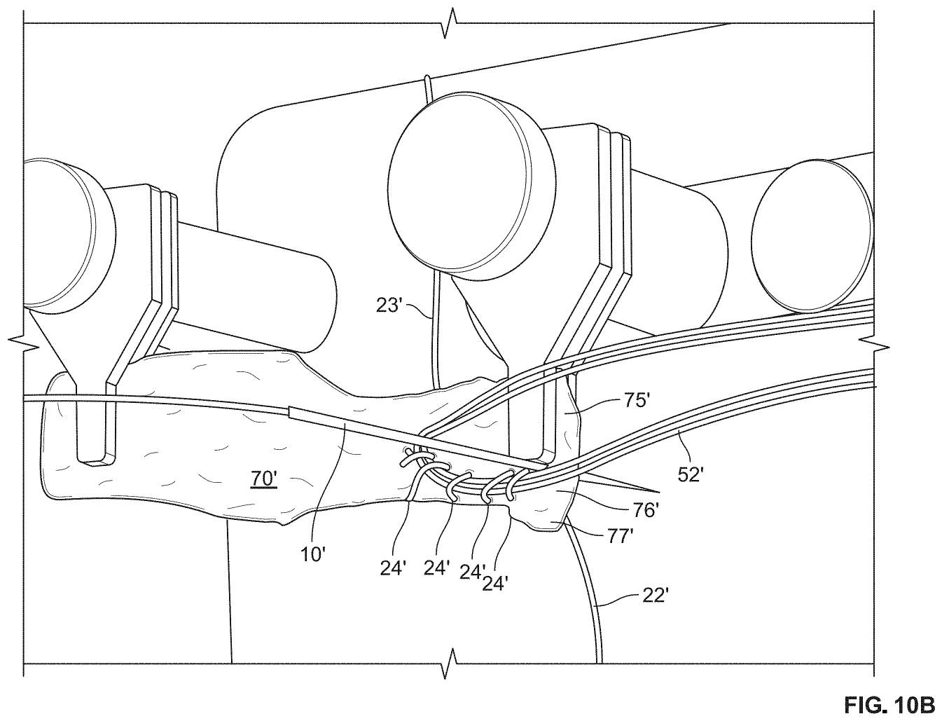

FIGS. 10A-10B are similar to FIGS. 4A-4B and will not be repeated here. Following the steps described as to FIGS. 4A-4B (and thus FIGS. 10A-10B), filament 20′ forms a running stitch pattern along the length of at least a portion of graft 70′ towards longitudinal end 75′ of graft 70′ and around the at least one loop 52′, in a first direction, illustrated as a counterclockwise direction along loop(s) 52′ from the luggage-tag configuration 25′ to the graft end 75′. As above, FIG. 10B illustrates how, as above as to FIG. 4B, the operator should include one or more passes, and as illustrated two passes, of length 22′ through, the lower, top and bottom corners 76′, 77′ of graft 70′ at end 75′. These final passes 24′ of length 22′ through these corners may assist in forming a tapered shape in the end 75′ of graft 70′.

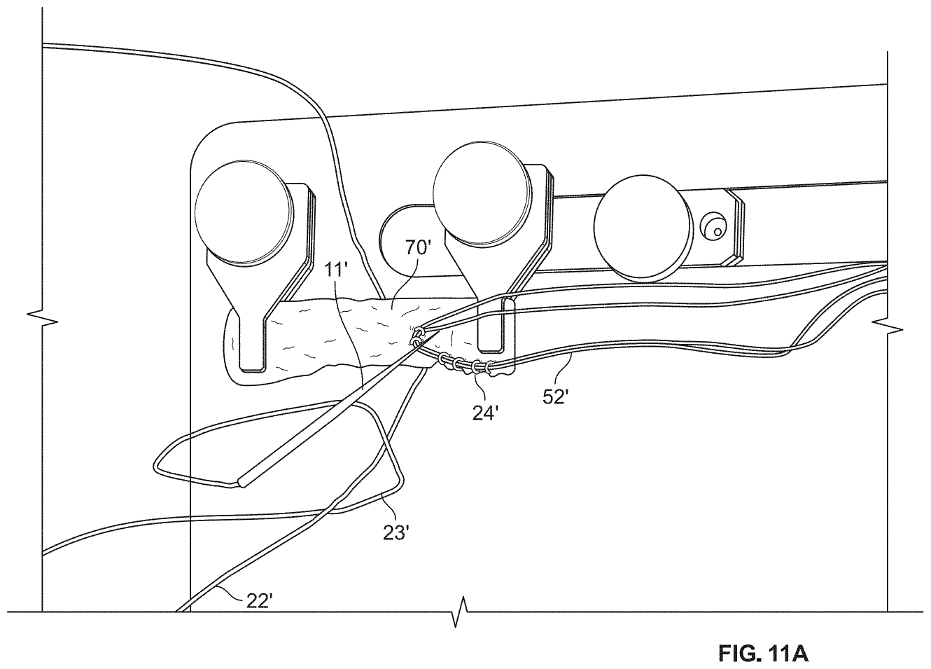

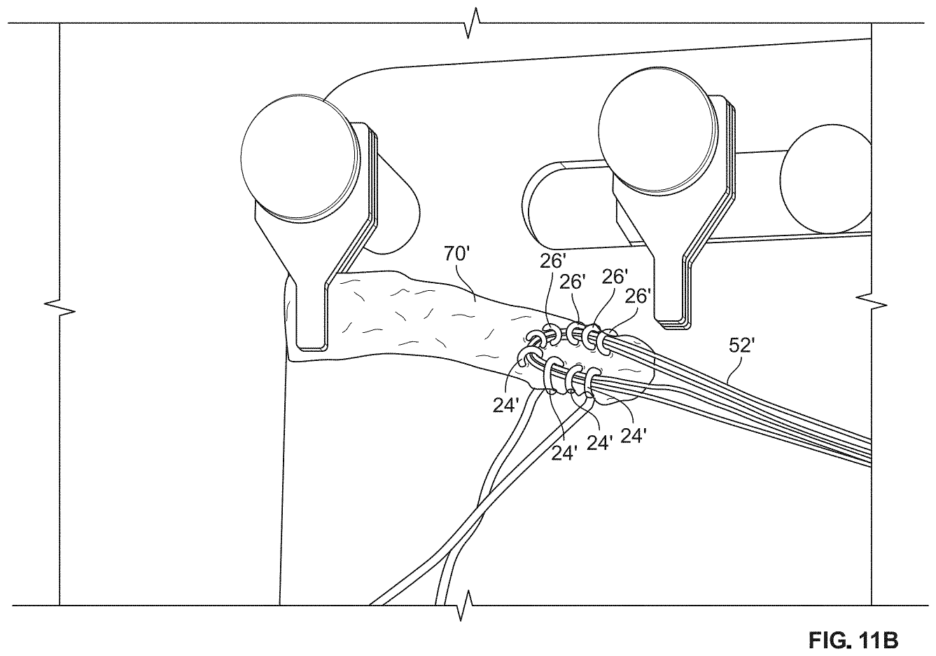

Following the step of FIG. 10B, this present embodiment starts to differ from the example discussed previously. In particular, rather than passing the length 22′ of filament 20′ back along the graft (as in FIGS. 4C-4D), needle 10′ is put aside for now, and instead, as illustrated in FIGS. 11A-11B, second needle 11′ is used to repeat the steps of FIGS. 10A-10B with needle 11′ and length 23′ of filament 20′ to form similar passes 26′ to create a running stitch pattern along the other lateral edge of graft 70′ and around the portion of the at least one adjustable loop 52′ that was not ensnared by the previous suturing in FIGS. 10A-10B, that is the stitch is formed in a second direction, illustrated as clockwise around the loop(s) 52′ from the luggage-tag configuration 25′. Once again, needle 11′ and associated length 23′ remain positioned at longitudinal end 75′ of graft 70′.

Continuing with this embodiment, FIGS. 12A-12D are representations of the implant system and soft tissue graft 70′ as positioned following the step of FIG. 11B. These illustrations also show a representative example of the running suture patterns formed by needle 10′ (spiral passes 24′) and needle 11′ (spiral passes 26′) discussed above as to FIGS. 10A-11B. The at least one adjustable loop 52′ is represented by two loops, though in some embodiments (including any of the embodiments discussed above) a simple continuous loop of suture or the like may be used, as discussed above. As lengths 22′, 23′ are tensioned (as discussed above, tensioned at each pass 24′, 26′ and/or at any time), the graft 70′ is tensioned through the continuous running stitch pattern to give the graft 70′ a tapered shape (FIG. 12B). In this embodiment, each length 22′, 23′ is shown to pass not just through the corners on its particular side of graft end 75′, but they also then pass through the graft at the opposite corner(s) or at least adjacent to the opposite corners. This creates a purse string configuration which may further assist compression of end 75′ to enunciate the tapering shape of graft 70′. It should be noted that, prior to the lengths 23, 24 being passed back up along graft 70 in the previous embodiment (as in FIGS. 4C-4D and 5C-5D), lengths 23, 24 can similarly be passed across the end 75 of graft 70 to the opposite corner(s) (or nearby the opposite corner(s)) such that filament 22 passes back up the graft 70 along the length where filament 23 first traveled down the graft 70, and vice versa on the other side of graft 70.

The ends of filament 20′ may then be secured, such as by forming a knot 27′ or the like, to secure filament 20′ and graft 70′ in position (FIG. 12C), similar to the knot or the like of FIG. 6A discussed above. This knot can then be buried into the body of the graft 70′ (FIG. 12D) similar to that discussed above as to FIG. 6B, and the tails of filament 20′ can be cut at a tendon-filament interface (formed upon the filament lengths 22′, 23′ exiting from the graft in the step of burying the knot). Alternatively, the tails may remain uncut and utilized for backup fixation. Finally, similar to that discussed above as to FIG. 6C, the graft 70′ can be removed from clamps 61′, 62′, if used, and the operator can test the at least one adjustable loop 52′ to ensure it can slide relative to graft 70′ and within the suture construct of filament 20′. For instance, the operator can pull on either end of at least one loop 52′, extending away from graft 70′, to ensure loop(s) 52′ can slide (FIG. 12E).

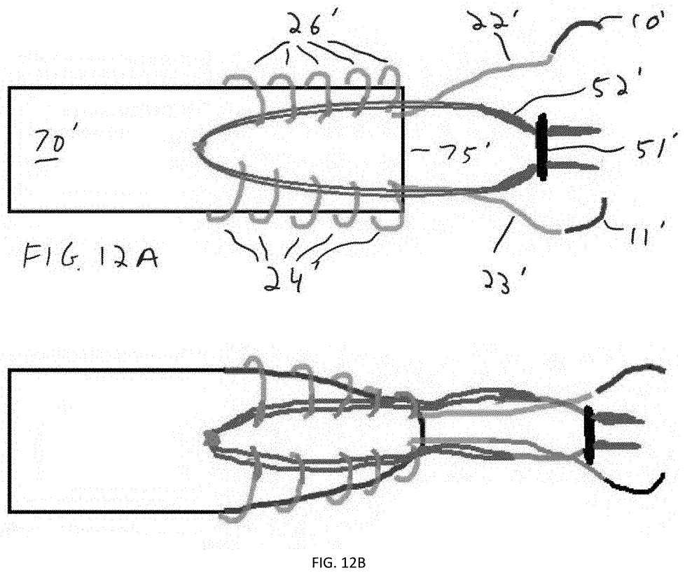

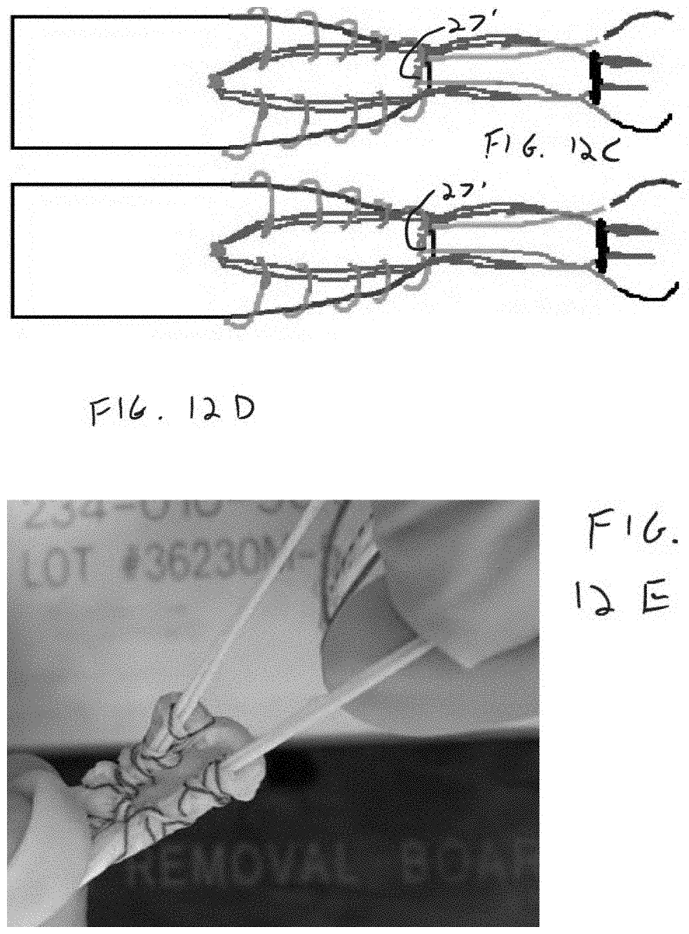

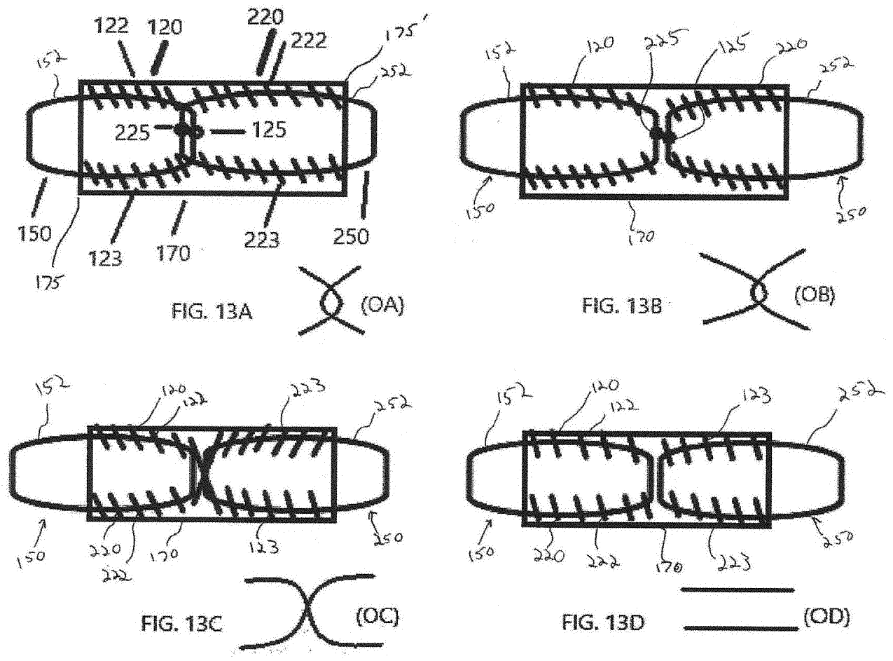

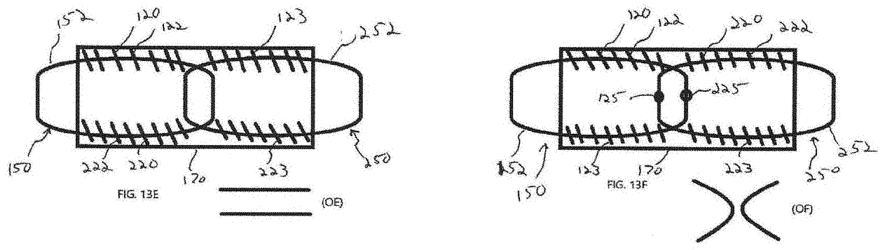

FIGS. 13A-13F illustrate still further exemplary embodiments of various methods of preparing a soft tissue graft, again utilizing an exemplary fixation system as that described above with reference to FIGS. 1A and 1B, though in this instance described using similar reference numbers including a length of filament 120 with filament lengths 122, 123, associated needles 110, 111 (not shown), and graft support filament 150. Graft support filament 150, as noted above, may be a ProCinch™. Additionally, in these examples, a second graft support filament 250 is also included, and may be a ProCinch™, or more specifically, may be a ProCinch™ Open Loop (Stryker Corporation, Greenwood Village, Colo.). For ease of illustration, FIGS. 13A-13F illustrate a simple oval for graft support filaments 150, 250, though as discussed above they each include adjustable loop(s) 152, 252, respectively, and fixation devices 151, 251, respectively.

Looking specifically at the exemplary embodiment of FIG. 13A, if the ProCinch™ Open Loop is used, then the filaments which eventually form adjustable loop(s) 252 of graft support filament 250 can be looped around the adjustable loop(s) 152 of graft support filament 150 such that the loops 152, 252 of both graft support filaments 150, 250 are interconnected with one another (also see a similar loop configuration in FIGS. 13E and 13F), such that fixation devices 151, 251 are positioned opposite one another adjacent opposite longitudinal ends 175, 175′ of soft tissue graft 170. Alternatively, loops 152, 252 (for any combination of ProCinch™ and/or ProCinch™ Open Loop used) can simply be connected to one another by, for instance, a separate piece of suture looped around the loops and knotted to hold them together. Still further, as discussed below, as illustrated in FIGS. 13B-13D, graft support filaments 150, 250 may simply be positioned near one another along soft tissue graft 170 and not directly connected to one another.

Continuing with FIG. 13A, and as detailed above, filament 120 may be connected to graft support filament 150 via a luggage tag 125 or the like, and similarly, filament 220 may be connected to graft support filament 250 via a luggage tag 225 or the like. Lengths 122, 123, 222, 223 of filaments 120, 220 may then be used to stitch the graft support filaments 150, 250 to soft tissue graft 170 as desired, such as by similar techniques as discussed above as to Exemplary Embodiments 1 and 2. The resulting stitching pattern of filaments 120, 220 are illustrated in FIG. 13A, and in the accompanying schematic representation, configuration “OA”, whereby the filaments 120, 220 cross over one another around the midsection of soft tissue 170, as do the graft support filaments 150, 250.

This illustrative embodiment of FIG. 13A, and similarly FIGS. 13E and 13F, with interconnected loops 152, 252 of opposing graft support filaments 150, 250, may assume a larger amount of the forces in the repair which would typically be undertaken by the soft tissue graft 170. Such a construct may have benefits in that the soft tissue graft may be better protected by the sutures during healing of the repair, and the sutures may even serve to protect the soft tissue graft in instances of extreme forces experienced at the repair. Subsequently, the soft tissue graft may undertake a greater amount of the load as tissue ingrowth occurs between the bones and the soft tissue graft. However, in some instances, the sutures (graft support filaments 150, 250 and/or filaments 120, 220) may continue undertaking most of the forces, particularly where ingrowth may not be as robust as intended, such that the graft could be just “along for the ride.” Nevertheless, even in these instances, the soft tissue graft may assist in re-establishing the integrity and natural anatomy of the joint, and their relationship to the graft support filaments 150, 250 and filaments 120, 220 can depend upon the material properties and construct properties of such filaments; for example the creep properties or absorbable properties may help in transitioning the forces in the joint from the suture construct to the soft tissue graft over time.

In another of these exemplary embodiments, illustrated in FIG. 13B, loop(s) 152, 252 are each again connected to soft tissue graft 170, but in this example the loops are not directly interconnected with one another as they were in the embodiment illustrated in FIG. 13A. Instead, the loops 152, 252 are simply arranged adjacent to one another along soft tissue graft 170. As illustrated, and as with the other illustrated embodiments, the graft support filaments 150, 250 are arranged such that the loop(s) 152, 252 extend along at least portions of soft tissue graft 170, and to and beyond respective ends 175, 175′ of graft 170. Beyond respective ends 170, 175′, each graft support filament 150, 250 may include a fixation device 151, 251 (not shown) such as a button implant as commonly known in the art for securing the graft 170 to other anatomy such as bone. However, while loop(s) 152, 252 do not overlap one another in this example, lengths of filament 120, 220 still cross over one another around the midsection of soft tissue 170, as illustrated by configuration “OB”. The stitching configuration “OB” may be accomplished by, for example, connecting filament 120 to graft support filament 250 via a luggage tag 125 or the like, and similarly, filament 220 may be connected to graft support filament 150 via a luggage tag 225 or the like. Lengths 122, 123 of filament 120, which is now connected to graft support filament 250, may then be stitched along loop(s) 152 of graft support filament 150. Similarly, length 222, 223 of filament 220, which is now connected to graft support filament 150, may then be stitched along loop(s) 252 of graft support filament 250.

As noted above, in FIG. 13B and similar examples herein, the graft support filaments 150, 250 are positioned close to one another along graft 170 such that they are close, but do not overlap, one another. That said, it may be beneficial to maintain some separation between the two graft support filaments 150, 250 to avoid having a large amount of suture at the mid-section of graft 170. On the other hand, one of the main benefits of this construct overall is to minimize the extension of the loop(s) 152, 252 from the end(s) of graft 170 to a fixation device (button, or the like). As such, it is important to find the balance between these two concepts to position the graft support filaments 150, 250 properly relative to one another and relative to the graft 170. For instance, on a soft tissue graft 170 of about 70 mm, separating the graft support filaments 150, 250 by a distance of about 30 mm may provide a beneficial balance of distributing suture along the mid-section of the graft 170, providing enough loop(s) 152, 252 structure along the graft 170 to ensure strong fixation between the loop(s) and the graft, and still minimizing the length of loop(s) 152, 252 beyond the ends of the graft 170 (which of course are adjustable in length if using a construct like ProCinch® or the like). One side-by-side comparison of such separation is illustrated in FIG. 13G, where the upper image is similar to the configuration of FIG. 13B, while the bottom image illustrates the two graft support filaments spaced from one another such that the mid-section of the graft includes less suture mass relative to the upper image. The dotted portions of lengths 122, 123, 222, 223 depict portions of the lengths extending underneath the graft, e.g., the dotted portions of lengths 222, 223 extend from the luggage tag 225, underneath the graft, and then form the stitching pattern extending along loops 252. The dotted portions of lengths 122, 123, 222, 223 could alternatively be positioned on the upper surface of graft, or even pass in and out of graft as desired. Such spacing of the loops could be included in other variations including those throughout FIGS. 13A-F and elsewhere disclosed herein.

In another example, FIG. 13C illustrates a similar configuration where the loop(s) 152, 252 are each connected to soft tissue graft 170, but as in FIG. 13B, the loops are not directly interconnected with one another. Lengths of filament 120, 220 are likewise not directly connected to either loop(s) 152, 252 (e.g., via a luggage tag or the like), but instead filament 120 is stitched along one side of loops 152 (length 122) and then one of the sides of loops 252 (length 123). Similarly, filament 220 is stitched along one side of loops 152 (length 222) and then one of the sides of loops 252 (length 223). As illustrated by orientation “OC”, filaments 120, 220 cross one another around the midportion of soft tissue graft 170 and around the area where graft support filaments 150, 250 are adjacent one another along graft 170. However, this crossover could happen anywhere, or multiple times, along the length of graft 170. For instance, length 122, instead of continuing across the top of graft support filament 150 as in FIG. 13C, could instead cross downward to the bottom of filament 150, and length 222 could cross upward from the bottom of filament 150 to the top of filament 150; further crisscrosses could then occur as desired.

FIG. 13D illustrates a variation of FIG. 13C where lengths of filament 120, 220 do not cross one another at all, such that filament 120 remains on the “top” of soft tissue graft 170 and graft support filaments 150, 250 while filament 220 remains on the “bottom” of soft tissue graft 170 and graft support filaments 150, 250. Configuration “OD” illustrates filaments 120, 220 as generally parallel to one another, such that they do not cross over one another.

In such configurations as in FIGS. 13C-13D, forces applied to the fixation system and soft tissue graft 170 may be transferred to the graft itself more than just the sutures of the fixation system. For instance, if forces are applied to the construct illustrated in FIGS. 13C-13D, the forces on loops 152 would tension loops and one or both filaments 120, 220 and then to at least a portion of the graft 170. Similarly, the forces on loops 252 would tension loops and one or both filaments 120, 220 and then to at least a portion of the graft 170. In this configuration, graft 170 may undertake a greater amount of the load since the loop(s) 152, 252 are not directly interconnected with one another, as may be the case in FIGS. 13A, where the loops are interconnected.

FIG. 13E illustrates an exemplary variation of the example of FIG. 13D, where graft support filaments 150, 250 are interconnected with one another. Configuration “OE” illustrates that in this instance, lengths of filament 120, 220 are in a similar configuration to configuration “OD” of FIG. 13D.

FIG. 13F illustrates an exemplary variation of the example of FIGS. 13A-13B where graft support filaments 150, 250 are interconnected with one another, like in FIG. 13A, but the lengths of filament 120, 220 are configured similar to that of FIG. 13B, e.g., by connecting filament 120 to graft support filament 250 via a luggage tag 125 or the like, while lengths 122, 123 are stitched along loop(s) 152 of graft support filament 150, and similarly, filament 220 may be connected to graft support filament 150 via a luggage tag 225 or the like, while lengths 222, 223 are stitched along loop(s) 252 of graft support filament 250. However, since graft support filaments 150, 250 are interconnected, filaments 120, 220 do not actually cross over one another, as evidenced by configuration “OF”, as opposed to configuration “OB” of FIG. 13B. Thus, in this example, loop(s) 152, 252 are interconnected but filaments 120, 220 do not overlap with one another.

The examples illustrated in FIGS. 13E-13F may benefit from the stability of having the graft support filaments interconnected (like in FIG. 13A), but also may benefit from the transfer of forces along more of the soft tissue graft 170 since filaments 120, 220 engage both graft support filaments instead of just one (like in FIGS. 13B-13D). Such configurations may have improved stability while also allowing for the graft itself to experience forces on the repaired joint of the patient. Such configurations may also lead to a more anatomically correct repair for the patient and improved graft maturation.

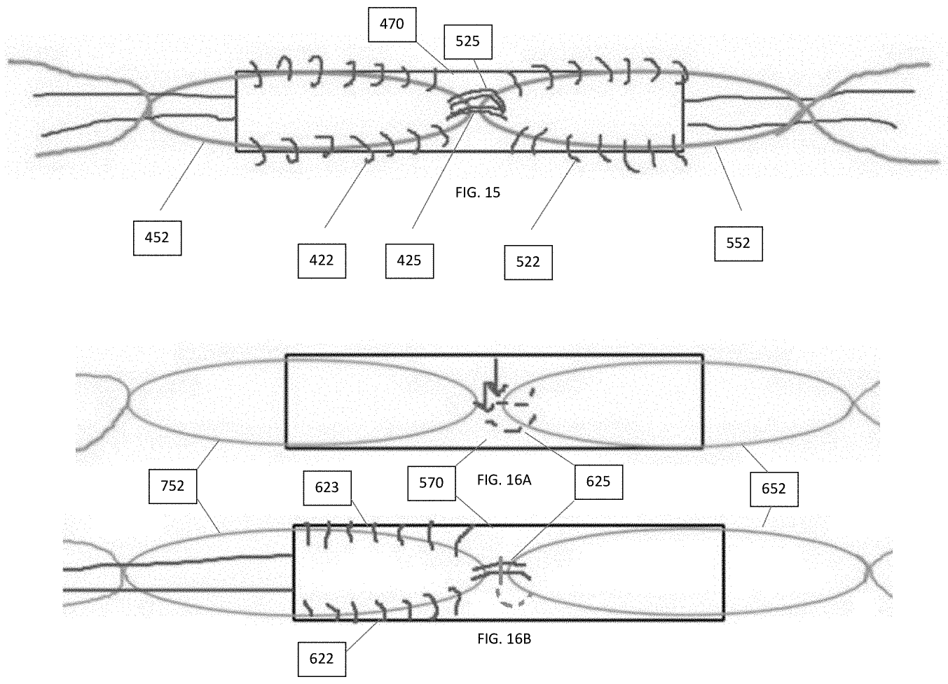

In yet another embodiment, FIG. 15 illustrates one example of loop(s) 452, 552 positioned on graft 470, which are connected via lengths of filament 420, 520 (including lengths 422, 522 by way of illustration); FIG. 15 includes similar numbers and references as in FIGS. 1-14, and thus reference numbers of similar tail number references similar structures unless otherwise indicated. The primary difference in this FIG. 15 versus the construct of FIG. 13B is that luggage tags 425, 525 (or the like) are each positioned around both loop(s) 452, 552, such that both luggage tags 425, 525 may create a connection between loop(s) 452 and loop(s) 552. Ends 422, 423 may extend from luggage tag 425 in any direction desired to stitch a portion of loop(s) 452 or loop(s) 552 to graft 470, and similarly, ends 522, 523 may extend from luggage tag 525 in any direction desired to stitch a portion of loop(s) 452 or loop(s) 552 to graft 470. As discussed elsewhere herein, the ends 422, 423, 522, 523 at an end of graft 470 may be knotted together or otherwise used to taper the particular end of graft 470, or may be used to do a return stitch back towards the luggage tags 425, 525. The ends of the two filaments positioned at each end of graft 470 may then be knotted together or otherwise secured, as discussed herein.

In still another embodiment, FIGS. 16A-B illustrate one example of loop(s) 652, 752 positioned on graft 570, which are connected via lengths of filament (filament 620, including lengths 622, 623, by way of illustration); FIGS. 16A-B include similar numbers and references as in FIGS. 1-15, and thus reference numbers of similar tail number references similar structures unless otherwise indicated. These FIGS. 16A-B are largely similar to the construct of FIG. 13B, with the primary difference being that no direct luggage tag is formed around loop(s) 652, but instead, ends 622, 623 are first passed through the graft 570 and then ensnare loop(s) 652 and are then passed over loop(s) 752 and immediately passed through graft 570. Such a construct may provide similar stability to other embodiments (e.g., which utilize a luggage tag configuration) but with reduced friction between filament 625 and loop(s) 652 due to the absence of a direct luggage tag. Ends 622, 623 are then used to stitch loop(s) to graft 570. Another length of filament (not shown) may do the same construct, but starting with ensnaring loop(s) 752 and ultimately stitching along loop(s) 652. The ends of the two filaments positioned at each end of graft 570 may then be knotted together or otherwise secured, as discussed herein.

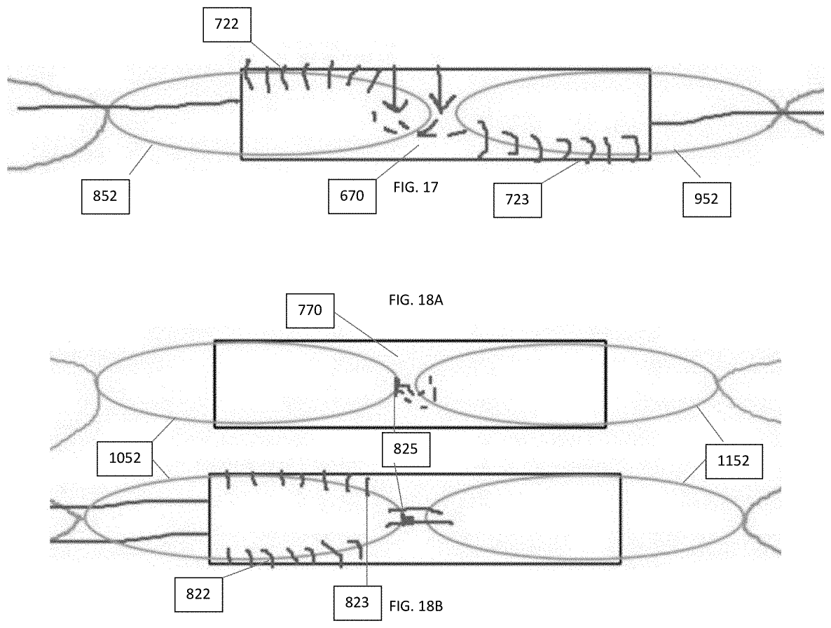

In another embodiment, FIG. 17 illustrates one example of loop(s) 852, 952 positioned on graft 670, which are connected via lengths of filament (filament 720, including lengths 722, 723, by way of illustration); FIG. 17 includes similar numbers and references as in FIGS. 1-16, and thus reference numbers of similar tail number references similar structures unless otherwise indicated. The ultimate construct of FIG. 17 is largely similar that of FIG. 13C, with the primary difference being that, filament 720 is introduced around one of loop(s) 852, 952 (illustrated around loop(s) 852), and ends 722, 723 crisscross along the underside of graft 670, before being used to stitch along loop(s) 852, 952. This loop and crisscross configuration replaces a luggage tag, knot or other feature discussed in other embodiments herein. A second length of filament (not shown) may be applied to this construct in similar fashion as filament 720 to stitch the remaining portions of loops(s) 852, 952 not stitched by filament 720. The ends of the two filaments positioned at each end of graft 670 may then be knotted together or otherwise secured, as discussed herein.

In a further embodiment, FIGS. 18A-B illustrate one example of loop(s) 1052, 1152 positioned on graft 770, which are connected via lengths of filament (filament 820, including lengths 822, 823, by way of illustration); FIGS. 18A-B include similar numbers and references as in FIGS. 1-17, and thus reference numbers of similar tail number references similar structures unless otherwise indicated. In this variation, filament 820 is connected to loop(s) 1052 via luggage tag 825 as discussed above. Ends 822, 823 extending from luggage tag 825 are then passed through graft 770 and then back up through graft again and through loop(s) 1152. Ends 822, 823 are then brought over both loop(s) 1152, 1052, passed through loop(s) 1052 and through graft 770 and then stitched along loop(s) 1052 as illustrated in FIG. 18B. As one or more points along the pathway of ends 822, 823, they may be crisscrossed to form additional structural rigidity and tension in the construct. A second length of filament (not shown) may be positioned in the same way as filament 820, but starting with a luggage tag on loop(s) 1152. The ends of the two filaments positioned at each end of graft 770 may then be knotted together or otherwise secured, as discussed herein.

An operator may balance these considerations with various suturing configurations using an at least one filament 120. Configurations such as those illustrated in FIGS. 13A-13F, and others similar to those, are envisioned and useful depending on the desires of the surgeon and the particular anatomy and needs of the patient. For instance, alternatives may include different stitching patterns than those illustrated above. For example, looking at FIGS. 13A, 13B and 13F, instead of lengths 122, 123 extending along one graft support filament 150/250 and lengths 222, 223 extending along the other filament 150/250, lengths 122, 222 may extend along one graft support filament 150/250 and the other lengths 123, 223 may extend along the other filament 150/250. Such configurations may be useful in certain instances for an operator seeking a particular load distribution along soft tissue graft 170 and graft support filaments 150, 250.

Alternatively or additionally, while each of graft support filaments 150, 250 are illustrated on the same side of the soft tissue graft 170, other configurations include, for example, where graft support filament 150 is on one side of soft tissue graft (e.g., the top surface of graft 170) while graft support filament 250 is on the other side of the soft tissue graft 170 (e.g., the bottom surface of graft 170). Since the lengths of filament 120, 220 typically pass through the entirety of the thickness of soft tissue graft 170 while performing the various stitching configurations, the graft support filaments 150, 250 can be on either side or face of graft 170, and can be on the same face of the graft 170 as one another (as illustrated throughout FIGS. 13A-F) or on opposite faces of the graft 170 from one another.

Still further, in other variations of the embodiments discussed and/or illustrated herein, the loop(s) 52, 52′, 152, 252, could be positioned in different orientations relative to the graft 70, 70′, 170. For instance, FIGS. 6C and 12E illustrate a construct with two loops 52, 52′, both of which are against one surface of graft 70, 70′. However, in some variations, one loop may be positioned against an upper surface of the graft while the other loop may be positioned against a bottom surface of the graft. Then, as described herein, the suture ends 22, 22′, 23, 23′ may be passed through graft and around both loops 52, 52′ such that the loops are compressed against opposing sides or faces of the graft. Further, as to FIGS. 13A-F, one or both graft support filaments 150, 250 could have such a configuration, where the two loops 152, 252 of one or both graft support filaments 150, 250 could be separated on opposing faces of the graft 170.

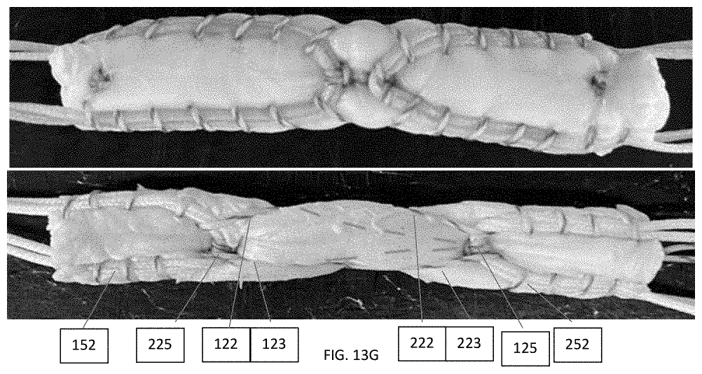

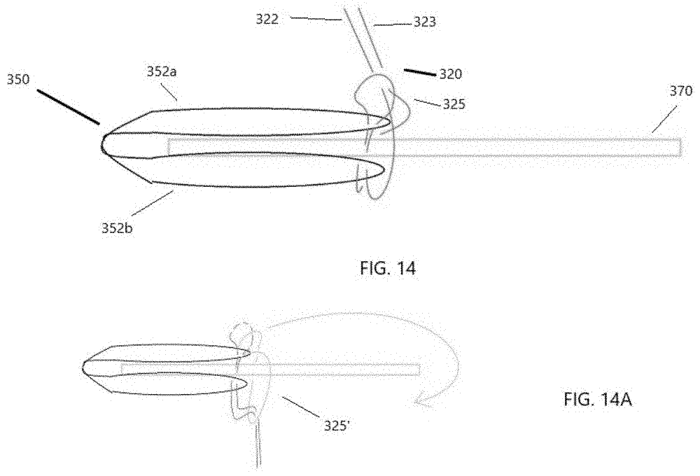

One such illustrative example of this configuration is in FIG. 14, which is a side view where, as with other embodiments illustrated and/or discussed herein, includes a graft support filament 350 positioned relative to a graft 370, though as noted above and illustrated in FIG. 14, in this embodiment of a filament 350 having two loops 352a, 352b, is positioned relative to the graft 350 such that loop 352a is on one surface of the graft (e.g., on the top surface of graft 370) while loop 352b is on the other surface of the graft (e.g., on the bottom surface of graft 370). A length of filament 320 may be used to connect filament 350 to graft 370, as discussed at length herein. In this instance, where a formed luggage tag 325 is illustrated, needles (not shown) on each of lengths 322, 323 are passed through loop 352a, into graft 370, and out through loop 352b. The needles may pass through graft at locations about 2-3 mm from one another, each closer to respective sides of the graft (e.g., in FIG. 14, one further into the page and one further away from the page). Length 323 is then brought around its respective side of graft 370 (e.g., out of the page) and length 322 is brought around its respective side (e.g., into the page) and then passed through a loop formed in filament 320 to achieve a luggage-tag configuration which can be tensioned to initially connect loops 352a, 352b to graft 370.

Lengths 322, 323 may be used to continue positioning loops 352a, 352b against respective faces of graft 370. For example, once the luggage tag 325 is created, the graft 370 may be unclamped (if already clamped as disclosed above) and flipped over (e.g., such that loop 352b is now on top of graft 370). Lengths 322, 323 may then be brought around respective sides of graft 370 and passed through loop 352b, through graft 370, and out through loop 352a. This process may be repeated as desired, and in any stitching configuration as taught herein or as desired. The resulting construct provides for a loop 352a, 352b on either side of the graft 370 which may provide a more balanced and symmetrical graft construct for implantation, especially considering the challenge of inserting a graft construct up through an oblique tunnel where if the loops 352a and 352b were on the same surface of the graft it might make the graft more likely to get caught on the edge of the oblique tunnel.

In one exemplary variation of FIG. 14, FIG. 14A illustrates an alternative method of forming the luggage tag configuration 325′ where, prior to passing ends 322, 323 through the loop of filament 320 to form the luggage tag 325, the loop of filament 320 may instead be passed around the end of graft 370 (see arrow of FIG. 14A) such that the loop now wraps around the sides of graft 370 and is positioned underneath the graft. Lengths 322, 323 are then passed through loop to form luggage tag 325′. The potential benefit of this method is that the graft does not need to be flipped over for subsequent stitching of the loops to the graft, but rather only one side of the graft need be unclamped (if previously clamped) to allow passage of the loop of filament 320 around the end of the graft.

In still further configurations, looking again at FIGS. 13A-13F for reference (but equally applicable to FIGS. 1-12 above), a graft support structure (not shown) could also be included along with graft support filaments 150, 250 to provide further strength to the prepared graft and implant system construct. For example, with both graft support filaments 150, 250 in FIGS. 13A-13F positioned on the top surface of soft tissue graft 170, the graft support structure could be positioned on the underside of graft 170. Further one or both lengths of filament 120, 220 could then, upon passing through soft tissue graft 170, also pass through the graft support structure one or more times to secure the graft support structure to the soft tissue graft 170 and filaments 120, 220. Such graft support structures may be a length of suture tape, a secondary graft construct (which could be formed of natural and/or artificial materials), or the like.

In still other configurations, a section or strip of biologic substance could be included along with graft support filaments (e.g., filaments 150), to provide a biologic benefit such as increased osteoconductivity. For example, a strip of Vitoss BA, Vitoss BA2X, or Vitoss BiModal (Stryker Corp., Allendale, N.J.) could be laid on the graft prior to executing any of the aforementioned stitch patterns. And still furthermore, a section or strip of material could serve both purposes of providing a biologic benefit along with a stitch reinforcement benefit, such as where the section or strip of material is a strip of suture tape coated with a biologic substance. In such instances, the length of filament (e.g., filament 120) can be passed through not only the soft tissue graft, but also through the section or strip of material.

In still other configurations, any of FIGS. 13A-13F (or the like) could also include a configuration where lengths of filament 120, 220 are stitched back in reverse along at least a portion of soft tissue graft, similar to that discussed above with reference to FIGS. 4D and 5D. Still further, during the stitching back, at least one of filament 122, 123, 222, 223 could perform at least one splice by passing through the body of one of filaments 120, 220. Such a splice could either be in the transverse direction or longitudinal direction. Such a splice provides added friction and locking power to further secure the whipstitching to the soft tissue graft 170. Of course, such splicing techniques could also be applied during the whipstitching performed in Exemplary Embodiments 1 and 2 (e.g., as in FIGS. 4D and 5D).

Moreover, the actual whipstitching pattern used may be any desired. While FIGS. 1-19, discussed herein, provide certain examples of whipstitching patterns, many other patterns and techniques are known in the art and may be applied to the soft tissue preparation methods disclosed herein. As noted above, a locking Krackow stitch may be such an alternative pattern or technique used. Other examples of whipstitching patterns using one or more lengths of filament are also envisioned.

FIG. 19 illustrates still another variation of connecting a graft support filament to a soft tissue graft. In this instance, as illustrated, a length of filament is connected to a graft support filament by a luggage tag (similar to for instance luggage tag 25, 25′, 125 of FIGS. 1A-1H, 7A-7H, 13A) or the like. The filament ends are then passed through the tissue (similar to for instance the configuration as in FIGS. 3A-3F and 9A-9F) such that the graft support filament 1252 and the luggage tag 925 abuts the soft tissue graft 870 (FIG. 19A). The length of filament ends 922, 923 then encircles the soft tissue graft and graft support filament, and is then secured by an overhand knot 928 or the like (FIG. 19B). This encircling step is then optionally repeated as desired, for example as illustrated in FIG. 19C where filament ends 922, 923 complete three encirclements. This configuration of filament ends 922, 923 may be helpful in making the graft more tubular or cylindrical in shape which more closely mimics the shape of the bone tunnel in which it may be at least partially positioned. Also as illustrated in FIG. 19C, at some point as the filament ends 922, 923 approach the end of the soft tissue graft 870, they may stop performing this encircling technique and revert back to a whipstitching pattern 924, 926 (similar to the whipstitching of FIGS. 12A, 13A, for example). This whipstitching at or near the ends of the graft help secure the filament to the graft since up to that point, the filament was only wrapped around the graft rather than passing through the graft. Upon completion of the whipstitching, the filament ends may be knotted 927 at or near the end of the graft (similar to FIG. 12C, for example) or the ends can be used to further whipstitch and/or encircle the soft tissue graft and graft support filament as desired, e.g., by traveling back in the opposite direction along the graft, the result of such a technique is illustrated in FIG. 6A, for example. Still further, the ends may be knotted or not, and instead of traveling back along the graft, the ends may instead be tied or otherwise connected to fixation device 51, 151, 252 for added securement of the graft and suture construct. As with other techniques and methods described above, this technique may be combined with other techniques disclosed herein. For instance, the method of encircling the tissue in this exemplary embodiment may be used during whipstitching disclosed in other embodiments, whether at the beginning, at the end, and/or intermittently, such as for example during the steps shown in FIGS. 4A and 5A.

Such embodiments as discussed above can be beneficial for soft tissue graft preparations, such as for ACL graft preparation, for a number of reasons. For instance, oftentimes when using adjustable loop structures to suspend an ACL graft (or the like), the loops must reside in between the fixation device and the graft, resulting in a long distance covered only by suture. This distance also must be positioned inside the bone tunnel, and thus the tunnel must be long enough to span this distance while still providing sufficient bone tunnel surface area to contact the soft tissue graft. The present disclosure provides a way to shorten this distance, allowing for a shorter bone tunnel while still maintaining the bone tunnel surface area. Specifically, as illustrated throughout, the graft resides alongside the adjustable loops rather than below the loops. In this configuration, it allows for some overlap of the soft tissue graft and the loop length (commonly referred to as the saddle length), and thus, allows for a shorter bone tunnel without sacrificing saddle length or interaction of the soft tissue graft with the bone tunnel.

Also, the present disclosure allows for a way to taper the graft, and upon tensioning of the suture and graft, the graft may take on a tubular shape, which is compatible with the cylindrical shape of the bone tunnel. Further, while typically a graft must be draped over and folded over a filamentary loop, the present disclosure allows for a single length of graft to be secured to the loop. This allows for a shorter graft, which is particularly important when an autograft is harvested from the patient's quad tendon or the like.

Further, as to any of the embodiments envisioned by the present disclosure, any of the filamentary materials utilized, such as length(s) of filament and/or graft support filament(s), may include a coating adapted to improve sliding of the filament relative to structures it contacts. Alternatively or in addition, such a coating may also be adapted to allow for tissue ingrowth. Such a suitable coating for promoting ingrowth may be hydroxyapatite powder or tricalcium phosphate, collagen-based additives, platelet-rich plasma, bioactive glass, or the like, any of which may be used depending on the type of tissue into which the filamentary material is being placed. One particular example includes a bioactive coating including bone graft particles (e.g., calcium phosphate, bioglass, or the like, or a combination thereof) and a polymer, such as polycaprolactone (PCL), as described in U.S. Pat. No. 10,729,548, the entirety of which is incorporated by reference herein. Similar such coatings are also described in U.S. application Ser. No. 16/906,460, the entirety of which is incorporated by reference herein.

Any of the filamentary materials may also include a coating adapted to promote healing in adjacent tissue or angiogenesis in the graft. Any of the filamentary materials may also include an at least one indicating marker along its length. Further, any of the filamentary materials may also include multiple colors or patterns along its length, for instance where a filament may include one color along a portion of the filament and a second color along another portion of the filament, wherein the colors may provide a distinguishing feature between the two portions of the filament.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method for preparing a soft tissue graft, comprising:
passing a suture material through the soft tissue graft adjacent to a graft support filament at a first longitudinal location along the graft support filament and the soft tissue graft, such that the suture material passes through the soft tissue graft on a first side of the graft support filament;
passing the suture material over the graft support filament and back through the soft tissue graft adjacent to the graft support filament at a second longitudinal location spaced longitudinally along the graft support filament and the soft tissue graft from the first longitudinal location, such that the suture material passes through the soft tissue graft on the first side of the graft support filament; and
tensioning the suture material to secure the graft support filament to the soft tissue graft along a length of the suture material between the first longitudinal location and the second longitudinal location.

2. The method of claim 1, wherein the suture material includes a needle, wherein during the passing steps the needle pierces the soft tissue graft to pass the suture material through the soft tissue graft.

3. The method of claim 1, wherein the passing steps and tensioning step are repeated at least twice at two more additional longitudinal locations along the graft support filament and the soft tissue graft.

4. The method of claim 1, wherein the graft support filament comprises a length of suture including at least one adjustable filamentary loop formed therein, wherein the first and second longitudinal locations are along a length of the at least one adjustable filamentary loop.

5. The method of claim 1, wherein, prior to the first passing step, the suture material is aligned and secured to the graft support filament such that the suture material and the graft support filament are manipulated as a single device relative to the soft tissue graft.

6. The method of claim 5, wherein, the suture material is aligned and secured to the graft support filament by folding the suture material on itself and securing the suture material to the graft support filament in a luggage-tag configuration, the method further comprising passing a first end of the suture material through the soft tissue graft, passing the second end of the suture material through the soft tissue graft, and pulling on the first end and the second end to position the luggage-tag configuration and the graft support filament against a surface of the soft tissue graft.

7. The method of claim 6, wherein the luggage-tag configuration is positioned at a location on the soft tissue graft distant from either end of the soft tissue graft, and the first longitudinal location is closer to one end of the soft tissue graft than the location of the luggage-tag configuration.

8. The method of claim 7, wherein the second longitudinal location is closer to the one end of the soft tissue graft than the first longitudinal location.

9. The method of claim 1, wherein, after the tensioning step, the graft support filament is slidable relative to the soft tissue graft and the suture material.

10. The method of claim 1, wherein the suture material comprises a first portion and a second portion, and the passing and tensioning steps use the first portion of the suture material, the method further comprising:
passing the second portion of the suture material through the soft tissue graft adjacent to the graft support filament at a third longitudinal location along the graft support filament and the soft tissue graft, such that the suture material passes through the soft tissue graft on the first side of the graft support filament, the third longitudinal location spaced laterally of the first and second longitudinal locations;
passing the second portion of the suture material over the graft support filament and back through the soft tissue graft adjacent to the graft support filament at a fourth longitudinal location spaced longitudinally along the graft support filament and the soft tissue graft from the third longitudinal location, such that the suture material passes through the soft tissue graft on the first side of the graft support filament; and
tensioning the suture material to secure the graft support filament to the soft tissue graft along a length of the suture material between the third longitudinal location and the fourth longitudinal location.

11. The method of claim 1, further comprising:
passing a second suture material through the soft tissue graft adjacent to a second graft support filament at a third longitudinal location along the second graft support filament and the soft tissue graft, spaced longitudinally from the first and second longitudinal locations, such that the second suture material passes through the soft tissue graft on a first side of the second graft support filament;
passing the second suture material over the second graft support filament and back through the soft tissue graft adjacent to the second graft support filament at a fourth longitudinal location spaced longitudinally along the second graft support filament and the soft tissue graft from the third longitudinal location, such that the suture material passes through the soft tissue graft on the first side of the second graft support filament; and
tensioning the second suture material to secure the second graft support filament to the soft tissue graft along a length of the second suture material between the third longitudinal location and the fourth longitudinal location.

12. A method of implanting a soft tissue graft in a bone tunnel formed in bone, comprising the steps of:
positioning the prepared soft tissue graft of claim 10 within the bone tunnel, prepared soft tissue graft including a fixation member connected to the graft support filament;
positioning the fixation member relative to the bone to secure the graft support filament relative to the bone; and
tensioning the graft support filament to position the soft tissue graft within the bone tunnel, wherein upon tensioning, at least a portion of the graft support filament slides relative to the soft tissue graft and the suture material.

13. The method of claim 11, wherein the graft support filament is positioned alongside the soft tissue graft and includes at least one adjustable filamentary loop, and the suture material is positioned to alternate between passing through the soft tissue graft and around the graft support filament along at least part of the length of the soft tissue graft and the graft support filament, wherein during the tensioning step, the at least one adjustable filamentary loop of the graft support filament slides alongside the soft tissue graft and through the suture material and the diameter of the at least one adjustable filamentary loop decreases.

14. A method of preparing a soft tissue graft, comprising:
positioning a first graft support filament having at least one adjustable filamentary loop at least partially against a soft tissue graft, such that at least a portion of the at least one adjustable filamentary loop of the first graft support filament extends beyond a first end of the soft tissue graft;
positioning a second graft support filament having at least one adjustable filamentary loop at least partially against the soft tissue graft, such that at least a portion of the at least one adjustable filamentary loop of the second graft support filament extends beyond a second end of the soft tissue graft;
securing the first graft support filament against the soft tissue graft such that the at least one adjustable filamentary loop of the first graft support filament is slidable relative to the soft tissue graft; and
securing the second graft support filament against the soft tissue graft such that the at least one adjustable filamentary loop of the second graft support filament is slidable relative to the soft tissue graft.

15. The method of claim 14, wherein the step of securing the first graft support filament against the soft tissue graft includes passing at least a first length of suture material through the soft tissue graft in at least a first location and passing around but not through the first graft support filament.

16. The method of claim 15, wherein the step of securing the second graft support filament against the soft tissue graft includes passing at least the first length of suture material through the soft tissue graft in at least a second location, spaced longitudinally from the first location, and passing around but not through the second graft support filament.

17. The method of claim 15, wherein the step of securing the second graft support filament against the soft tissue graft includes passing at least a second length of suture material through the soft tissue graft in at least a second location, spaced longitudinally from the first location, and passing around but not through the second graft support filament.

18. The method of claim 17, wherein the first length of suture material passes around the first graft support filament and not the second graft support filament, and the second length of suture material passes around the second graft support filament and not the first graft support filament.

19. The method of claim 17, wherein prior to the positioning steps, connecting the first length of suture material to the first graft support filament and connecting the second length of suture material to the second graft support filament.

20. The method of claim 14, wherein prior to the positioning steps, interconnecting the at least one filamentary loop of the first graft support filament with the at least one filamentary loop of the second graft support filament.
